(19) United States
(12) Patent Application Publication
(10) Pub. No.: US 2023/0302183 A1
(43) Pub. Date: Sep. 28, 2023

(54) LOW PROFILE LIGHT DELIVERY SYSTEM

(71) Applicant: UV Partners, Inc., Grand Haven, MI (US)

(72) Inventors: Michael Halloran, Hudsonville, MI (US); Ryan D. Schamper, Grand Haven, MI (US); Colin J. Moore, Grand Rapids, MI (US); David W. Baarman, Fennville, MI (US); David Alan Jerovsek, Grand Haven, MI (US)

(21) Appl. No.: 18/103,736

(22) Filed: Jan. 31, 2023

Related U.S. Application Data
(60) Provisional application No. 63/394,811, filed on Aug. 3, 2022.
(60) Provisional application No. 63/305,080, filed on Jan. 31, 2022.

(51) Int. Cl. A61L 2/24; A61L 2/10

(52) U.S. Cl. CPC A61L 2/24; A61L 2/10; A61L 2202/11; A61L 2202/14

(57) ABSTRACT

A low profile disinfection and delivery system that utilizes a channel lighting system with a parabolic multifunctional anisotropic reflector for effectively distributing the power of UV LEDs. The channel lighting system delivers specific energy patterns to the target surface. The channel lighting system can be provided as a more integrated UV delivery system. The homogenous delivery and distribution of patterned energy enables better use and efficiency of the available UV LED power.

### BACKGROUND OF THE INVENTION

The present invention relates to light deliver systems, and more particularly to systems for providing controlled distribution of light. The present invention is well-suited for use in the distribution of all types of light, including without limitation UV light and visible light.

Some embodiments of the present invention relate to the use of UV light to treat surfaces that are touched more frequently.

Inactivation of microorganisms on contaminated surfaces (target surface) using ultraviolet (UV) light is driven by the UV dose delivered to that surface. Dose, in this context, refers to the product of the radiant flux (intensity, W/m2) and the time of exposure. It therefore follows that the time needed to adequately treat a target surface is inversely proportional to the intensity of UV light delivered to that surface.

In the ideal case, all of the energy from a UV light source (such as a lamp or LED) would be evenly distributed over the target surface. This even distribution would ensure the entire surface was disinfected in the minimum possible time. In such a scenario, a modest power source could quickly disinfect a large surface. In practice, it is exceedingly difficult to achieve this uniform distribution. Power sources such as lamps and LEDs instead create areas of high intensity (hot spots) which can introduce issues, while other areas of the target surface have low intensity (cold spots) that require longer treatment times in order to reach the necessary dose. This distribution problem can be mitigated by using a higher number of lower power light sources and/or advanced optics that evenly diffuse the UV light. Both of these alternatives can be prohibitively expensive, however, and involve significant redesigns of the core product systems (mechanical, electrical, and thermal) for every different size of target surface.

Distribution of energy can be significantly enhanced by moving the light source further away from the target surface, but this solution is often undesirable for consumers for both aesthetic and practical reasons. Fixturing the light source at an appropriate distance can be awkward, as the light may block the field of view or motion of operators who are trying to use the target surface. Past system required high angles to the treated surface and a higher number of LEDs to cover a specific area. Other sources like cold cathode, dielectric barrier discharge (DBD), or low pressure mercury create difficult issues related to size and surface angles.

### SUMMARY OF THE INVENTION

The present disclosure provides an apparatus that can distribute energy from one or more light emitting diodes (LEDs) over a target surface area (e.g., on the order of square feet, such as the approximate size of many surfaces or kiosk touchscreen devices) while maintaining, in the context of UV light distribution, a highly uniform UV intensity field and a low profile (e.g., a 10 mm protrusion located on one side of the target surface). Moreover, this device is highly customizable, with different sizes of target surface being treatable via adjustment, such as by adjusting one or more of the orientation of an LED in the system, an angle of the device relative to the target surface, and another configurable parameter. One embodiment of the present disclosure provides a UV source that casts energy onto a longitudinal parabolic reflector with anisotropic properties. The measured UV energy levels are sufficient to treat the surface at between 2 and 10 uW/cm². This data illustrates how the anisotropic reflector enables extending the power and expanding the available energy from the source energy.

In the context of UV treatment, the present disclosure effectively provides a solution that deactivates pathogens quickly reducing the transmission of disease. Embodiments of the present disclosure can utilize a controller to control dosages between uses of the target surface, e.g., between sales at a self-checkout kiosk. In some embodiments, the computer system associated with the kiosk can send a message to the energy distribution system so it can activate based on the state of the system associated with the target system, e.g., once a sale is complete or a transaction is complete the kiosk computer can send a message to the UV device. For example, the message can be communicated to the UV device controller for processing or the message can be a control signal communicated to the LED driver directly. The controller may be configured to identify and act based upon one or more inputs from occupancy sensors, time of flight sensors, accelerometry, or other sensors for automatic shut off override or other features. By receiving messages, sensor input, or a combination thereof, the system can essentially understand when a new sale is or has taken place and can be configured to enable UV energy cycles to be driven specifically when suitably between sales or transactions.

The UV device or module can be configured to mount at a suitable angular position and height to the target surface. For example, the UV device or portion thereof can be configured to be held by fasteners at a suitable angular position and height to the target surface. A low surface angle can provide a desirable energy distribution across the target surface and enable easier and simpler integration directly into multiple treatment solutions. Further, directing the UV energy while creating a specific pattern allows the more, if not substantially all, of the potential of the LED source to be utilized.

The disinfection cycle timing can be configured based upon the desired dosage to be provided. For example, a 10 uW/cm² dosage can be configured with a disinfection timing cycle shorter than a 2 uW/cm² dosage. In some disinfection solutions of the current embodiment, a 265 nm UVC LED is utilized, but this technology can apply generally across the UV spectrum. Cold cathode, dielectric barrier discharge (DBD), 222 nm LEDs and other sources can be utilized in conjunction with this technology. Further, antimicrobials can be combined with UV treatment to provide additional benefits. This technology can also benefit visible lighting configurations beyond the UV range for non-disinfecting lighting solutions.

These and other objects, advantages, and features of the invention will be more fully understood and appreciated by reference to the description of the current embodiment and the drawings.

Before the embodiments of the invention are explained in detail, it is to be understood that the invention is not limited to the details of operation or to the details of construction and the arrangement of the components set forth in the following description or illustrated in the drawings. The invention may be implemented in various other embodiments and of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components. Any reference to claim elements as “at least one of X, Y and Z” is meant to include any one of X, Y or Z individually, and any combination of X, Y and Z, for example, X, Y, Z; X, Y; X, Z ; and Y, Z.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a 3D plot of UV power delivered to an area by casting a UV energy pattern from an LED onto a longitudinal parabolic reflector with anisotropic properties.

FIG. 2 illustrates a modeled UV energy distribution (according to the provided color legend) using an exemplary anisotropic reflector with a single light emitting diode (“LED”) and parabolic configuration on a 20″ display (e.g., kiosk).

FIGS. 3A and 3B show front and side views respectively of an exemplary low-profile delivery system.

FIG. 4 shows a portion of an energy pattern on a surface of power delivered from the same low profile parabolic longitudinal channel of FIGS. 3A-B using a spectral reflector.

FIG. 5 shows a modeled energy distribution using an exemplary spectral reflector in the channel LED longitudinal parabolic configuration using a single 100 mW LED.

FIG. 6 shows a measured 3D plot of UV power delivered to an area by casting UV energy from a single LED onto a longitudinal parabolic reflector with known diffuse properties, such as Polytetrafluoroethylene (PTFE).

FIG. 7 shows a modeled energy distribution using a diffuse reflector in the channel LED longitudinal parabolic configuration using a single 100 mW.

FIG. 8 shows a position index for a UV light engine delivering UV energy to a target surface.

FIGS. 9A-D illustrate an exemplary embodiment of a mechanical UV reflector assembly.

FIGS. 10A-D show one embodiment of a mechanical relationship between a parabolic reflector and a light-emitting diode (LED) printed circuit board assembly (PCBA) in accordance with one embodiment of the present disclosure.

FIG. 11 shows an example of diffuse reflectors used on a 40″ display and a modelling of UV energy distribution.

FIGS. 12A-B show plots of UV energy from a low profile UV delivery system with variable UV LED power.

FIG. 13 shows a UV energy plot of another embodiment of a low profile UV delivery system in accordance with one embodiment.

FIGS. 14A-B show front and perspective side views of an elevated low profile UV delivery system in accordance with one embodiment.

FIG. 15 shows a front view of another elevated low profile UV delivery system in accordance with one embodiment.

FIG. 16 shows a perspective side view of a low profile UV delivery system in accordance with one embodiment emphasizing the angular relationship of the reflector to the display surface.

FIGS. 17A-C show an example of modeling multiple spectral segments combined to make a larger parabolic reflector.

FIG. 18A illustrates a profile view of a parabolic reflector and portion of a display.

FIG. 18B illustrates a front view of a display with the parabolic reflector of FIG. 18A installed along the bottom edge of the display.

FIGS. 19A-B show front and side views of a 40″ touch display with a channel UV LED system using a parabolic reflector and two 100 mW UV LEDs.

FIG. 20 shows a modeled UV energy distribution plot of delivered energy to the display of FIGS. 19A-B.

FIG. 21 shows an end of an exemplary UV channel of the present disclosure looking down the parabolic anisotropic reflector.

FIGS. 22A-B show perspective and top views of an anisotropic reflector in accordance with one embodiment.

FIGS. 23A-B show an example of two reflectors positioned end to end with each reflector having three different reflector portions.

FIG. 24 shows an exemplary anisotropic reflector.

FIG. 25 shows another exemplary anisotropic reflector.

FIGS. 26A-B show perspective and side views of two anisotropic reflectors positioned end to end in another exemplary configuration.

FIG. 27 shows an exemplary block diagram of a low profile UV delivery system.

FIG. 28 shows the same system as FIG. 27 but configured to treat the right side of the display.

FIG. 29 shows another configuration of the FIG. 27 system.

FIGS. 30A-C shows an example of a telescoping treatment unit that can expand to different sizes to treat different display area sizes.

FIG. 31 shows the target focal point on the parabolic reflector for a 10 degree beam angle LED.

FIGS. 32A-B shows top and end views of one embodiment of a low profile UV delivery system.

FIGS. 33A-C show one embodiment of a low profile UV delivery system with louvers in both horizontal and vertical axis.

FIG. 34 shows a table of calculations for on time and LED life in one embodiment.

FIG. 35 shows the integrated UV channel system within an airport check in and ticketing kiosk to disinfect those high traffic surfaces.

FIG. 36 shows a representation of an integrated UV channel system utilized within a keyboard to treat the surface using the kiosk like control and treatment system

FIG. 37 shows a representation of an integrated UV channel system in a laptop display to disinfect the keyboard surface when the display is at specific angles that can treat or dose the keyboard properly.

FIG. 38 shows a representation of an integrated UV channel system within a portable point of sale system to treat the high traffic surface.

FIG. 39 shows a representation of an integrated waterproof UV channel system within an ATM to disinfect those high traffic surfaces.

FIG. 40 shows a representation of an integrated UV channel system within a retail POS system to treat those high traffic surfaces.

FIG. 41 shows a representation of an integrated UV channel system within a food preparation table to disinfect those high traffic surfaces.

FIG. 42 shows a representation of an integrated UV channel system within a medical cart treating the monitor, the keyboard and the work surface to disinfect those high traffic surfaces. The low profile nature allows treatment in small spaces like keyboard storage areas.

FIG. 43 is a perspective view of a portion of a channel reflector in accordance with an embodiment of the present invention.

FIG. 44 is a perspective view of a portion of a channel reflector with representative surface texturing.

FIG. 45 is a representation sectional view taken transversely across the channel reflector with representative light rays.

FIG. 46 is a representation sectional view take taken longitudinally along a portion of the length of the channel reflector with representative light rays.

FIG. 47 is a perspective view of a channel lighting system in accordance with an embodiment of the present invention.

FIG. 48 is a perspective view of parabolic reflector with an alternative distal end configuration.

FIG. 49 is a schematic representational side view of an LCD illumination system in accordance with an embodiment of the present invention.

FIG. 50 is a schematic representational top view of the LCD illumination system of FIG. 49.

FIG. 51 is a schematic representational side view of an LCD illumination system in accordance with an alternative embodiment of the present invention.

FIG. 52 is a schematic representational top view of the LCD illumination system of FIG. 51.

FIG. 53 is a schematic representational top view of another alternative LCD illumination system.

FIG. 54 is a schematic representational side view of an alternative LCD illumination system incorporating a separate diffuser.

FIG. 55 is a schematic representational top view of the LCD illumination system of FIG. 54.

FIG. 56 illustrates an exemplary UV treatment pattern generated by an embodiment of the low profile UV light delivery system.

FIG. 57 illustrates another exemplary UV treatment pattern generated by an embodiment of the low profile UV light delivery system.

FIG. 58 illustrates exemplary reflected light beam divergence from one embodiment of a low profile UV delivery system.

FIG. 59 illustrates reflector geometry including relative position and size of a reflector and light source to generate the reflected light beam divergence of FIG. 58.

FIG. 60 illustrates exemplary reflected light beam divergence from one embodiment of a low profile UV delivery system.

FIG. 61 illustrates reflector geometry including relative position and size of a reflector and light source to generate the reflected light beam divergence of FIG. 60.

FIG. 62 illustrates a perspective representative model view of one embodiment of a low profile light delivery system with two light sources.

FIG. 63 illustrates a YZ-view of the low profile light delivery system of FIG. 62.

FIG. 64 illustrates an isometric view of the low profile light delivery system of FIG. 62.

FIG. 65 illustrates an XY-view of the low profile light delivery system of FIG. 62.

FIG. 66 illustrates another XY-view of the low profile light delivery system.

### DESCRIPTION OF THE CURRENT EMBODIMENTS

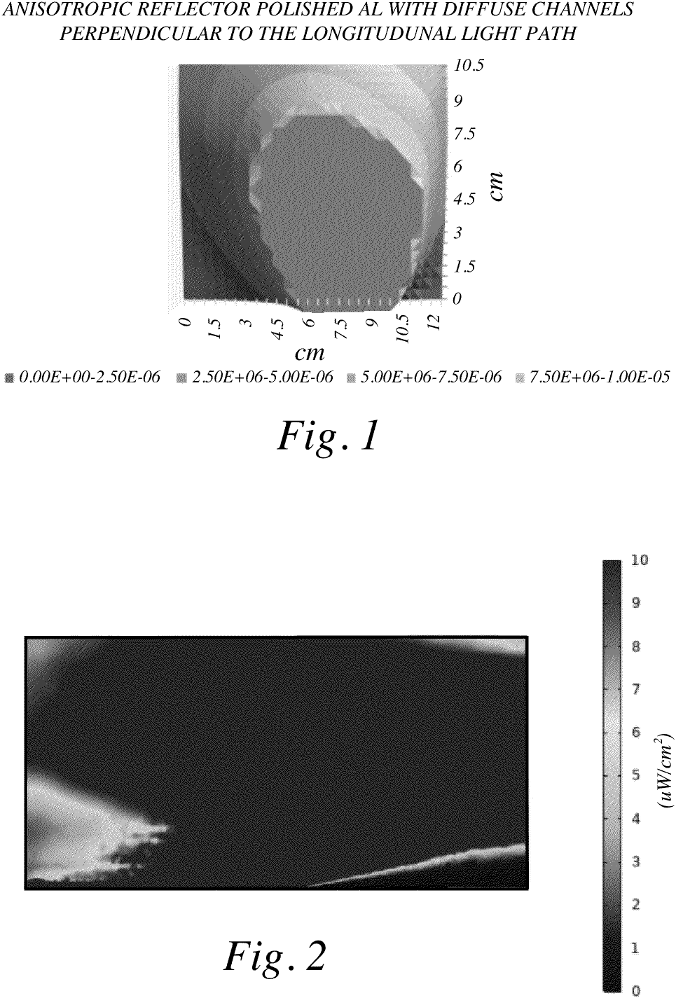

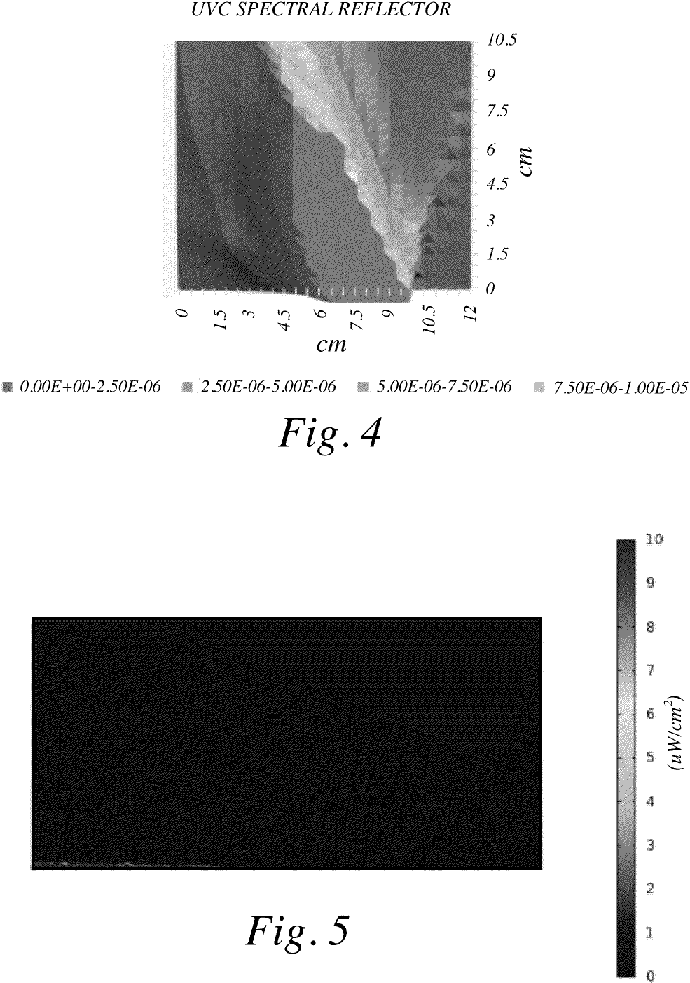

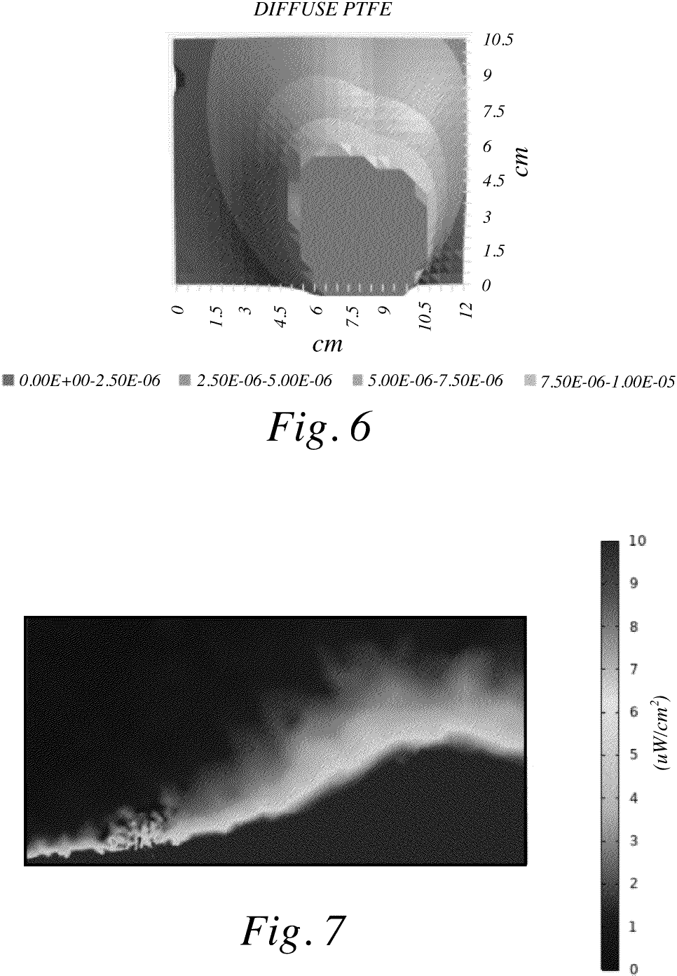

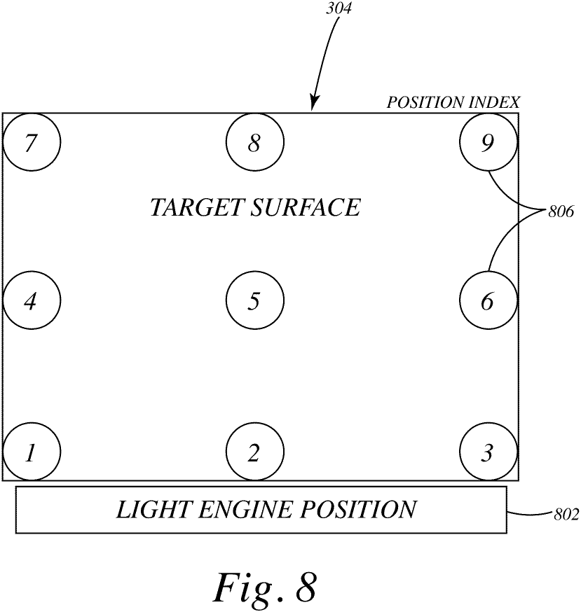

A 3D plot of UV power delivered to an area by casting a UV energy pattern from an exemplary single LED onto a longitudinal parabolic reflector with anisotropic properties is illustrated in FIG. 1. The measured UV energy levels are sufficient to treat a surface at between about 2 and 10 uW/cm². This figure illustrates how one embodiment of an anisotropic reflector enables extending the power and expanding the available energy from the source energy. The x-axis and y-axis represent two distance dimensions (e.g., width and height respectively) of an area in centimeters (e.g., a surface) and the z-axis (coming out of the page) shows the 3D measure UV energy according to the legend. It should be understood that the region centered at about 7.5 cm in the x-axis and 4.5 cm in the y-axis is where the 3D measurement was cut off and is a separate 3D energy measurement from the corners. The same is the case with the other 3D measured UV energy graphs shown in FIGS. 4 and 6.

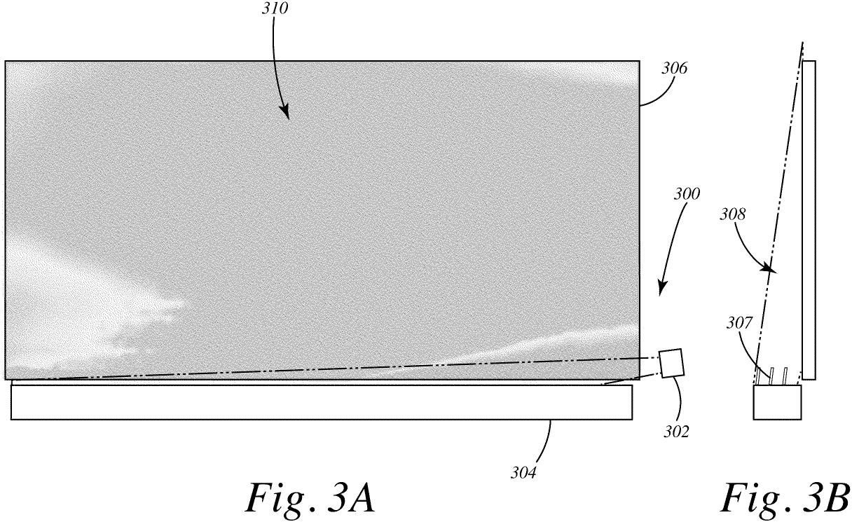

The first inventive aspect of this disclosure involves utilizing the power available in an LED over a larger surface by first directing LED beam into a longitudinal parabolic reflector at an angle related to the beam, parabolic length and target area to treat. The angle may, in some applications, be a slight 1-degree angle. FIGS. 3A and 3B show front and side views respectively of an exemplary low-profile delivery system. The depicted configuration utilizes a single 100 mW UV LED that delivers about 10 uW/cm2 to the majority of the 20″ touch screen surface. FIG. 2 illustrates a modeled UV energy distribution (according to the provided color legend) of the exemplary low-profile delivery system of FIGS. 3A-B.

The low-profile delivery system 300 of FIGS. 3A-B includes a UV light source 302 (e.g., an LED that provides a 10 degree beam of UV energy) and a multi-effect anisotropic reflector channel 304. The anisotropic reflector channel 304 can provide a projection window (e.g., having a 3-10 mm height) to cast UV energy 308 onto a display 306 in the form of a UV energy pattern 310. The channel is disposed along the bottom edge of the display 306, but in other embodiments the channel can be disposed along a different edge of the display. The channel 304 may provide protective louvers 307. In the current embodiment, the display is a 9 inch tall by 18 inch wide touch screen display (also referred to as a 20 inch touch screen due to the diagonal distance of the display).

The second inventive aspect of the disclosure involves using an anisotropic reflector to further distribute the power of an LED over a surface. The anisotropic reflector includes a UV reflective component (e.g., specular or spectral), a diffuse or diffusing component in one direction, and a spectral component in two directions perpendicular to the first direction. This allows the beam to be controlled and shaped in the second two directions, while scattering the energy uniformly in the third direction. This creates planes of energy that can be cast to target locations. The parabolic reflector may also have an angular element to cast energy back into the system. The exemplary embodiments are generally focused on utilizing one or two LEDs to treat large areas utilizing the power of the LED, which allows lower cost and simpler systems. Of course, the UV channel systems described herein can be extended to three, four, or more LED systems.

FIG. 4 shows a 3D UV energy plot of UV energy delivered to a portion of the surface from the same low profile parabolic longitudinal channel of FIG. 3 except using a spectral reflector. The x, y, and z axes of the plot are the same as FIG. 1. FIG. 5 shows a modeled energy distribution using an exemplary spectral reflector in the channel LED longitudinal parabolic configuration using a single 100 mW LED. The color according to the legend shows the amount of accumulated variable intensity of UV energy over the length and width of the display.

FIG. 6 shows a measured 3D plot of UV power delivered to an area by casting UV energy from a single LED onto a longitudinal parabolic reflector with known diffuse properties, such as Polytetrafluoroethylene (PTFE). PTFE can diffuse UV energy optically. FIG. 7 shows a modeled energy distribution using a diffuse reflector in the channel LED longitudinal parabolic configuration using a single 100 mW to deliver UV energy to a 20 inch kiosk display.

The third inventive aspect of the disclosure involves the sale or transaction cycle that drives the disinfection cycle from the transaction computer. In general, from a keyboard, it can be difficult for a system to identify when one person is complete, and another starts. With a kiosk, the transaction timing is easier to identify. For example, transaction timing can be associated with known trigger events such as when a payment is complete, a ticket has been issued, or a credit card has been accepted. The anisotropic features can be added to the reflector by creating a specific amount of ridges by roughing the surface in a specific direction. For example, this can be accomplished using a light brush of 150 Grit sandpaper assuring a good amount of spectral remains.

The fourth inventive aspect of this disclosure involves the use of multiple LEDs to cover a larger surface. FIG. 8 illustrates a position index for a UV light engine 802 delivering UV energy to a target surface 804. In the depicted figure UV energy measurements for nine different positions 806 referenced by numbers 1-9.

Table 1 (reproduced below) shows a compiled table of energy distribution over an exemplary target surface. A combination of elements including spectral and diffuse components can enable better performance by the anisotropic reflector assembly. The anisotropic diffuse spectral reflector provides a more homogenous distribution and better relative energy levels at the farther distances of the target surface (i.e., locations 7, 8 and 9). The anisotropic reflector performs better for this type of UV delivery enabling better use and distribution from a UV LED. Moreover, by integrating these reflector types by segments and by ratios the distribution including homogeneity of energy over the target surface can be managed.

| Location | Anisotropic | Diffuse | Specular | No Reflector |
|---|---|---|---|---|
| 1 | 1.32E-06 | 8.43E-07 | 1.49E-06 | 6.14E-07 |
| 2 | 4.95E-05 | 2.69E-05 | 2.02E-05 | 1.01E-05 |
| 3 | 7.48E-06 | 7.32E-06 | 8.88E-08 | 1.82E-07 |
| 4 | 5.76E-06 | 2.78E-06 | 3.44E-06 | 1.55E-06 |
| 5 | 2.03E-05 | 1.12E-05 | 1.49E-05 | 4.22E-06 |
| 6 | 1.17E-05 | 7.19E-06 | 9.20E-07 | 6.94E-08 |
| 7 | 5.25E-06 | 2.78E-06 | 3.94E-06 | 1.36E-06 |
| 8 | 7.98E-06 | 4.80E-06 | 2.89E-06 | 1.60E-06 |
| 9 | 5.34E-06 | 3.54E-06 | 3.72E-07 | 3.59E-08 |

The fifth inventive aspect of this disclosure involves varying the power to vary the dosage. This can be to save LED life knowing the cycle times at that time of day are longer or it can be varied by the anticipated cycle times per day saving LED life. When the kiosk becomes busy the dosage can be configured to increase dosage relative to activity, or go to a maximum suitable dosage.

The sixth inventive aspect of this disclosure relates to treating a surface zonally based on the touch areas being addressed. The kiosk computer generally understands (e.g., has data stored in memory indicative of) what zone the graphic payment (or other) touch buttons are located while finalizing the order. Combining this information with the ability to treat the surface zonally allows for UV energy to be cast as needed and planned for a balanced and longer LED life. The kiosk computer also can track the top selections for various times of day and adjust zonal treatment. Further, the graphic locations can be dynamically placed in different treatment zones for maximum LED life or balancing LED life for that system. Much like a display rotating graphics as to not burn the screen this is about balancing LEDs and passing information as to utilize the longest LED treatment performance of the system.

The seventh inventive aspect of this disclosure relates to tracking performance data and disinfection data in the cloud by either the kiosk computer or the disinfection controller. For example, the system can track treatment cycles, cycle times, and partial treatments.

The eighth inventive aspect of this disclosure relates to easy replacement of the LED PCBA at the ends of the parabolic reflector held with two screws at the desired angle. The PCBA holds the LED at the proper position and angle for dose delivery.

The ninth inventive aspect of this disclosure relates to the horizontal louvers and eyebrow to assist assure the low angle delivery of the UV energy. Vertical louvers can be placed to limit nodes of energy and further deliver the energy to the surface. The side facing the screen may be angled toward the screen and may be UV reflective returning the escaping energy back. The customer facing side of the louver is configured to be a blocker limiting the energy escaping (not delivered to the surface) the system.

The tenth inventive aspect of this disclosure relates to a sliding or telescoping variable length UV channel system. The UV channel system can be adaptive allowing configurability to a variety of different sizes of display surfaces. A telescoping or sliding UV channel can be a more compact, portable, and adaptive solution for aftermarket displays.

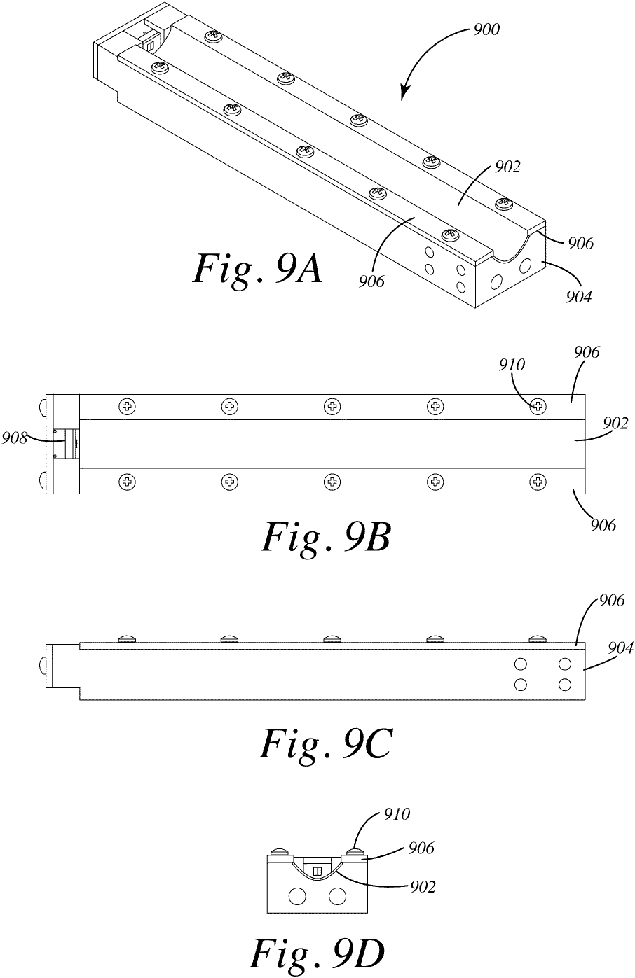

FIGS. 9A-D illustrates an exemplary embodiment of a mechanical UV reflector assembly 900. The reflector assembly 900 includes a channel body 904 that can include a parabolic reflector 902. The parabolic reflector can be integral with or joined to the channel body 904. A UV transmissive window or covering (not shown) can be retained by retaining ledges 906 and be held in place to the channel body 904 by screws 910. The channel body 904 can provide a 1 degree (or other specified) angle for mounting the LED assembly 908 at a specific position and orientation relative to the parabolic reflector. This configuration allows easy replacement of the LED assembly.

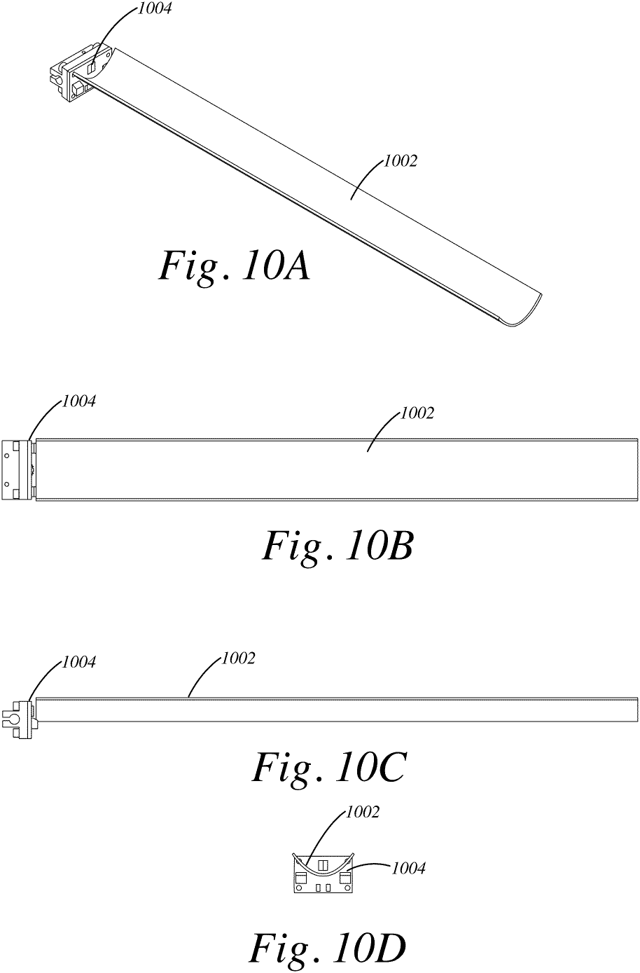

FIGS. 10A-D shows one embodiment of a mechanical relationship between a parabolic reflector 1002 and a light-emitting diode (LED) printed circuit board assembly (PCBA) 1004 in accordance with one embodiment of the present disclosure. The PCBA 1004 is shown in its angular relationship to the reflector. The angular relationship between UV LED, its beam angle and the parabolic reflector ultimately enables a desired distribution of UV LED energy across a target surface.

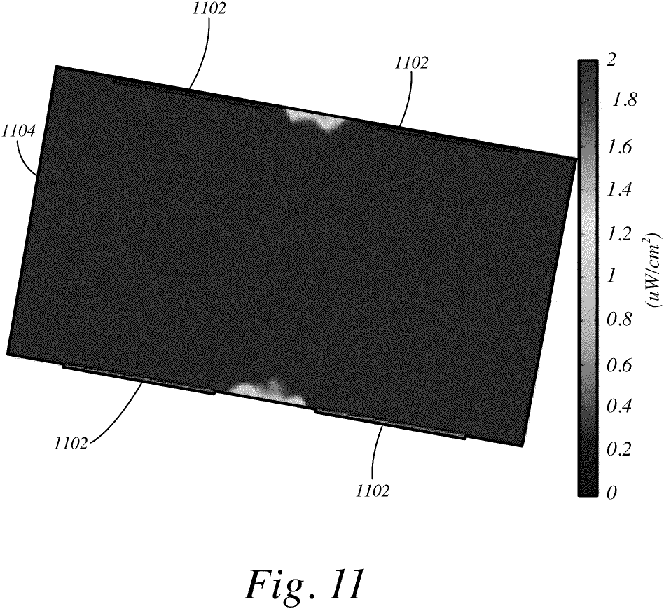

Several configurations of different exemplary embodiments of parabolic reflectors casting UV energy onto a display are illustrated in FIGS. 11-15.

FIG. 11 shows an example of diffuse reflectors 1102 used on a 40″ display 1104 and a modelling of UV energy distribution over the 40″ display. This embodiment uses four separate parabolic reflectors 1102 that each include one UV LED (not shown). The positions of the parabolic reflectors 1102 allows essentially complete coverage of the surface at 2 mW/cm².

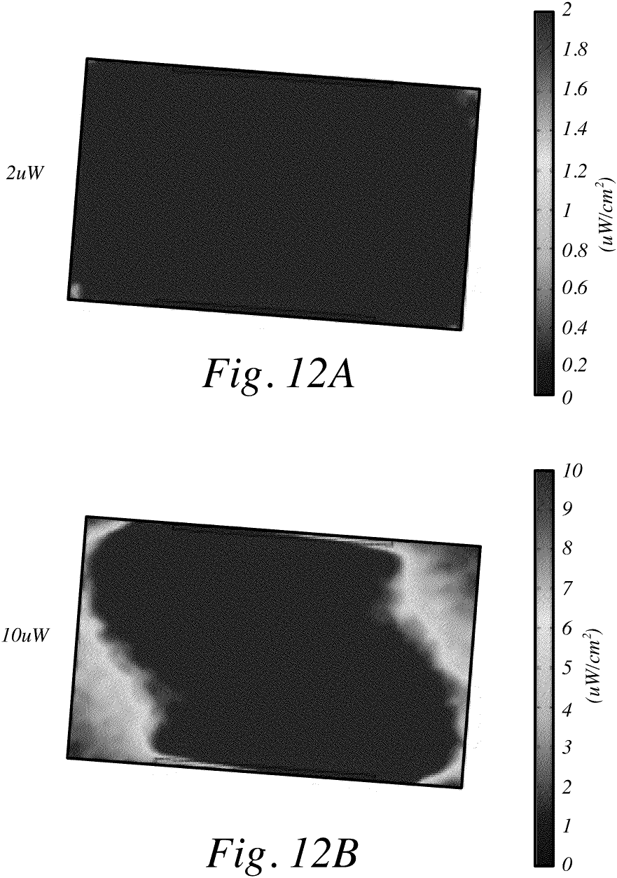

FIGS. 12A-B shows a 20″ kiosk 1204 with two reflectors 1202 and UV LED assemblies disposed along the top and bottom of the kiosk display. By varying the UV LED power the same 40″ surface can be treated at an intensity of 2 uW/cm2 (see FIG. 12A) or 10 uW/cm² (FIG. 12B).

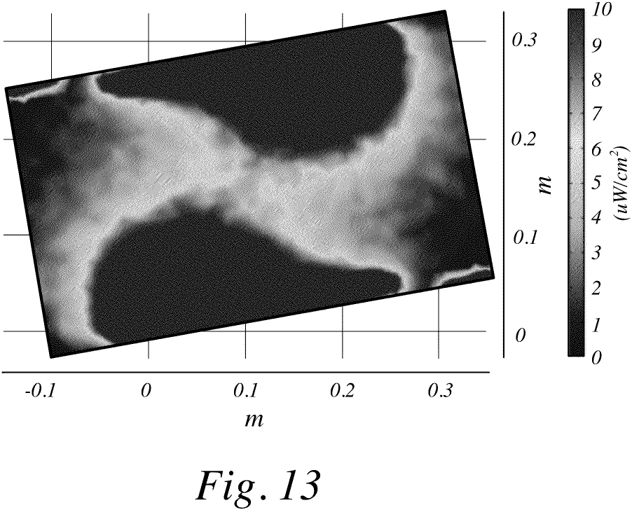

FIG. 13 shows a representation of delivered energy to a 21″ kiosk (18″ x 11″) display 1302 utilizing top and bottom reflectors (not shown). The reflectors in this embodiment are U shaped channels comprised of three 0.25” walls. The material of the reflectors for this embodiment is a 94% reflective diffuse reflector on vertical wall, 90% reflective specular reflectors on horizontal walls. The LED power for the illustrated plot was 100 mW and a single LED was utilized with each of the reflectors. The reflector height is configured such that the bottom of the reflectors is on the kiosk surface.

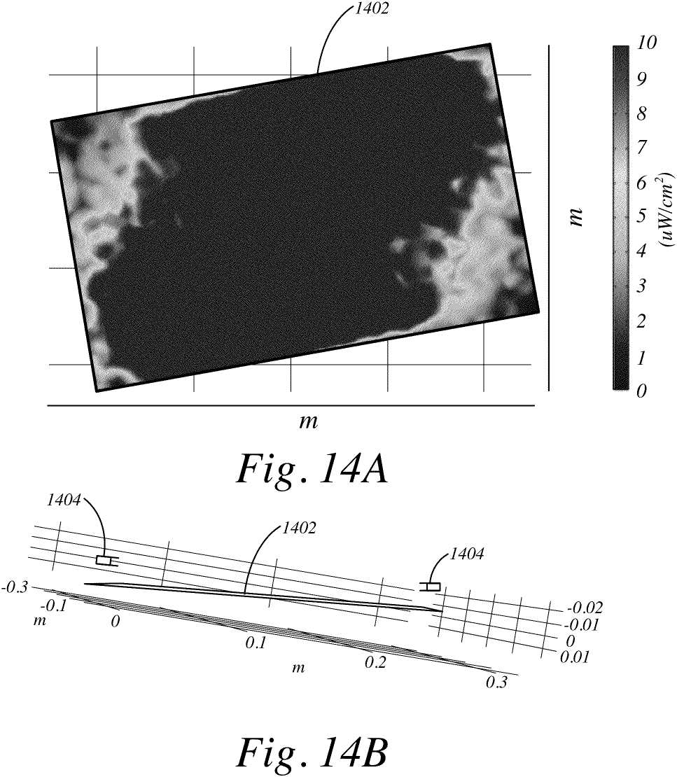

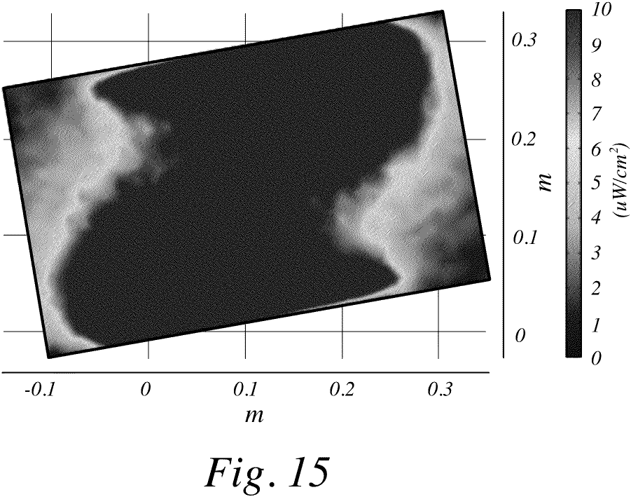

FIGS. 14A-B show the same reflector as used in FIG. 13 but elevated by about .5 inches above the kiosk surface to improve performance to 10 uW/cm² for the majority of the surface. FIG. 14B shows the spatial relationship between the display 1402 and the two reflectors 1404. FIG. 15 shows the same system as FIG. 13 and FIGS. 14A-B but where the reflectors are positioned at a height of 0.25” above the kiosk.

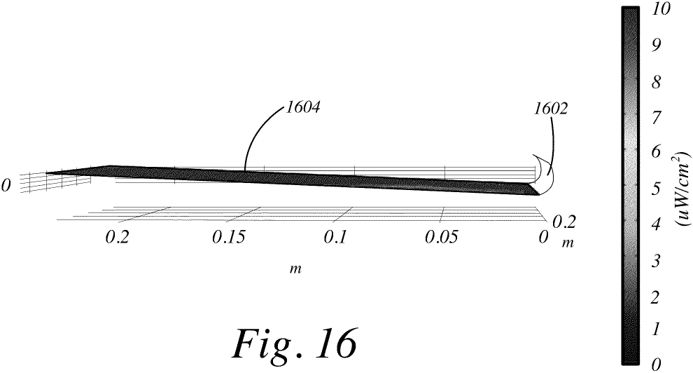

FIG. 16 shows the angular relationship of an exemplary reflector 1602 to an exemplary display surface 1604. In this embodiment, one UV LED with its power set to about 100 mW ultimately projects UV light onto a 20″ kiosk display 1604 (18″ x 19″) from an anisotropic reflector 1602 in accordance with an embodiment of the present invention. In the depicted embodiment, the low profile UV delivery system is the same as the one discussed in connection with FIG. 2. The angle between the reflector and the kiosk is:

$$\text{θ} = {sin}^{- 1} \left(\text{h}\right| \text{L}$$

The h symbol refers to the height of the reflector and the L symbol refers to the length of the Kiosk from the reflector the far edge. The shape of the reflector is a parabola that is 10 mm across, 3.5 mm deep, and 11 mm high. The LED is located along the central axis of the parabola with a focal point of:

$$\text{f} = \frac{{\left(\frac{\text{ω}}{2}\right)}^{2}}{4 \text{α}}$$

The focal point is provided in terms of LED height above the bottom of the parabola. The w symbol refers to the width of the parabola and the a symbol refers to the depth of the parabola. The reflector height is such that the bottom of the reflector is on the kiosk surface.

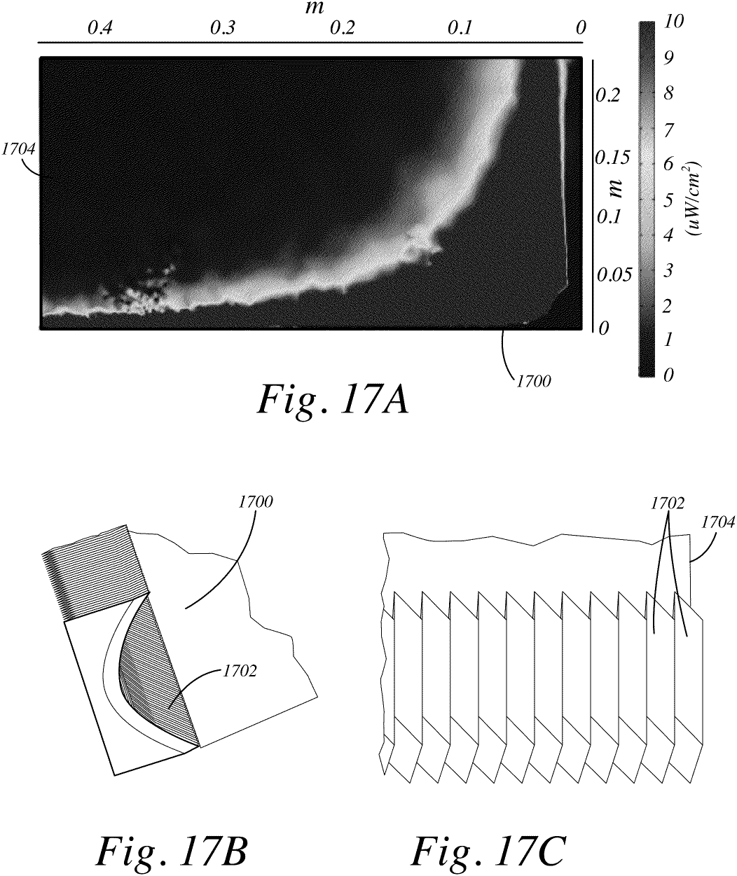

FIGS. 17A-C show an example of modeling multiple spectral segments combined to make a larger parabolic reflector. Specular segments 1702 include a parabolic face extruded at an angle determined by the segment dimensions. In the current embodiment, the segment sizes are 1 mm x 1 mm. In this embodiment, the parabolic reflector is positioned along the bottom edge of the display and the LED is positioned at the bottom right of the display oriented into the segmented parabolic reflector 1700.

show exemplary modeling parameters to obtain both specular and diffuse performance enabling 10 uW/cm² over a target surface. The reflector is anisotropic, causing collimation in the xy-plane (the result of specular reflection), while resulting in a diffuse reflection pattern in the yz-plane.

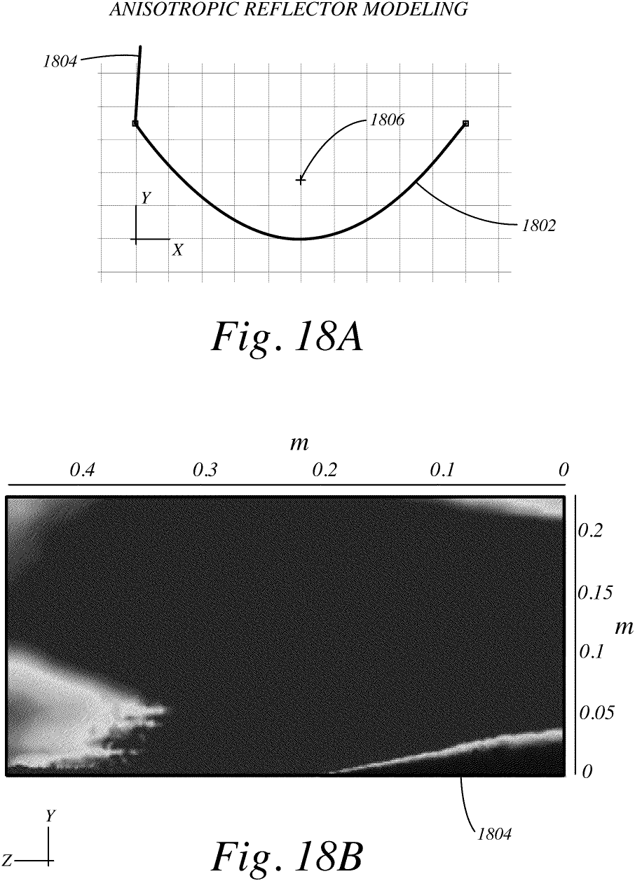

FIGS. 18A-B show exemplary modeling parameters to obtain specular and diffuse performance enabling 10 uW/cm2 over a target surface. FIG. 18A illustrates a profile view of a parabolic reflector and portion of a display and FIG. 18B illustrates a front view of a display with the parabolic reflector of FIG. 18A i3nstalled along the bottom edge of the display. Referring to FIG. 18A, the parabolic reflector 1802 receives UV light from the UV light source 1806, which is reflected toward the display surface 1804. Specifically, UV light rays emanate from the UV LED and intersect the anisotropic reflector. Upon reflection, the x-component of ray velocity is set to 0 (representing a specular parabola’s collimating properties in the xy plane). The y- and z-components of ray velocity are randomly selected from a probability distribution that represents diffuse reflection in the yz plane.

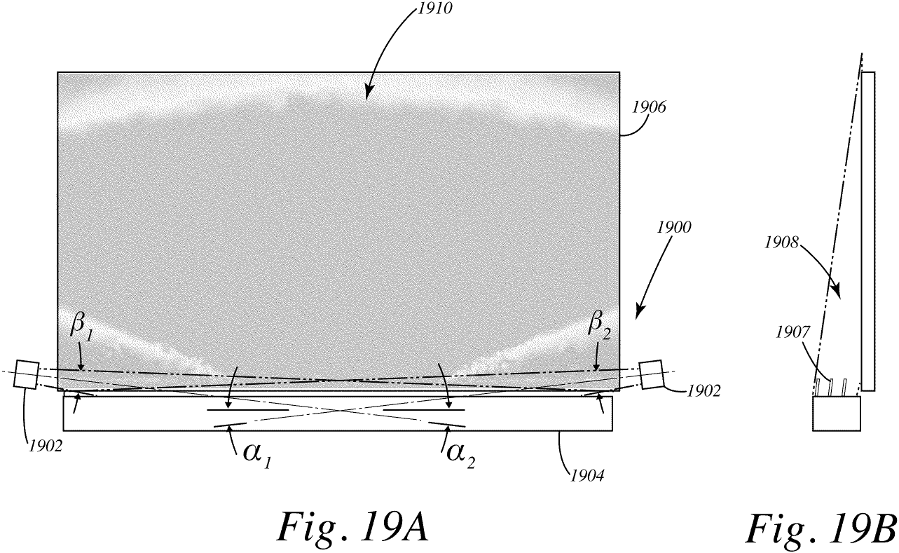

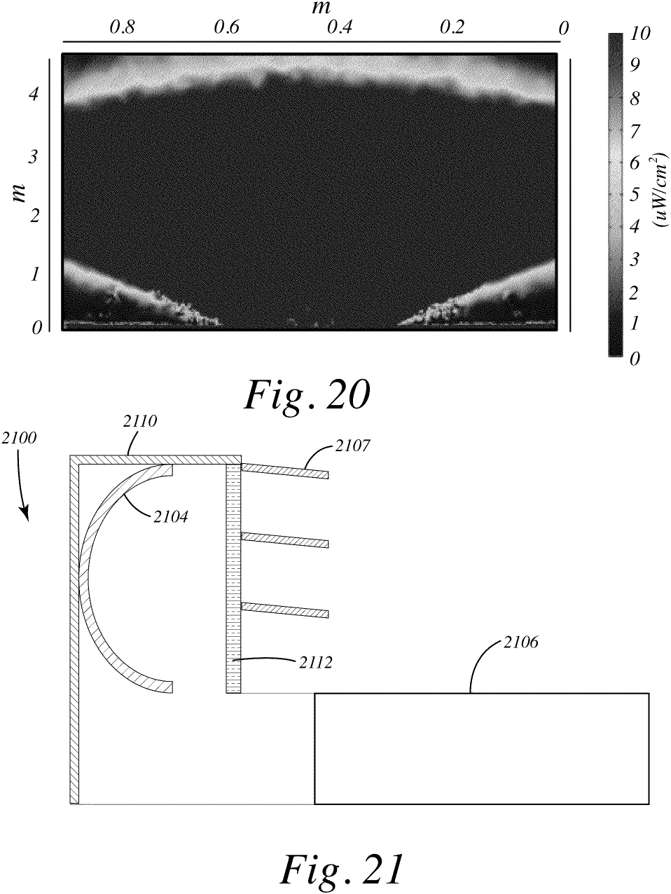

FIGS. 19A-B show front and side views respectively of a low profile UV delivery system for a display. In this embodiment, a channel UV LED system (i.e., a low profile UV delivery system) using a parabolic reflector and two 100 mW UV LEDs covers the majority of the surface with UV energy at 10 uW/cm2. The low-profile delivery system 1900 of FIGS. 19A-B includes two UV light sources 1902 (e.g., LEDs that each provide about a 10 degree beam of UV energy α₁, α₂) and a multi-effect anisotropic reflector channel 1904. The anisotropic reflector channel 1904 can provide a projection window to cast UV energy 1908 onto a display 1906 in the form of a UV energy pattern 1910. The channel is disposed along the bottom edge of the display 1906, but in other embodiments the channel and LEDs can be disposed along a different edge of the display. The channel 1904 may provide protective louvers 1907. In the current embodiment, the display is a 40″ display. FIG. 20 shows a modeled UV energy distribution plot of delivered energy to the display of FIGS. 19A-B, that is a 40″ kiosk with an anisotropic reflector having two LEDs.

FIG. 21 shows an end of an exemplary UV channel of the present disclosure looking down the parabolic anisotropic reflector. The low profile UV delivery system 2100 generally includes a parabolic reflector 2104 mounted within a lighting module housing 2110 that can fasten to or otherwise be associated with the display at a particular position and orientation. Louvers 2107 can be provided to assist in directing the UV energy to the surface and reduce the energy released beyond the delivery plane limiting exposure outside the energy path to the display. A transmissive window 2112 can be provided that permits UV energy to pass-through and cast upon the display module 2106 while protecting the reflector from debris.

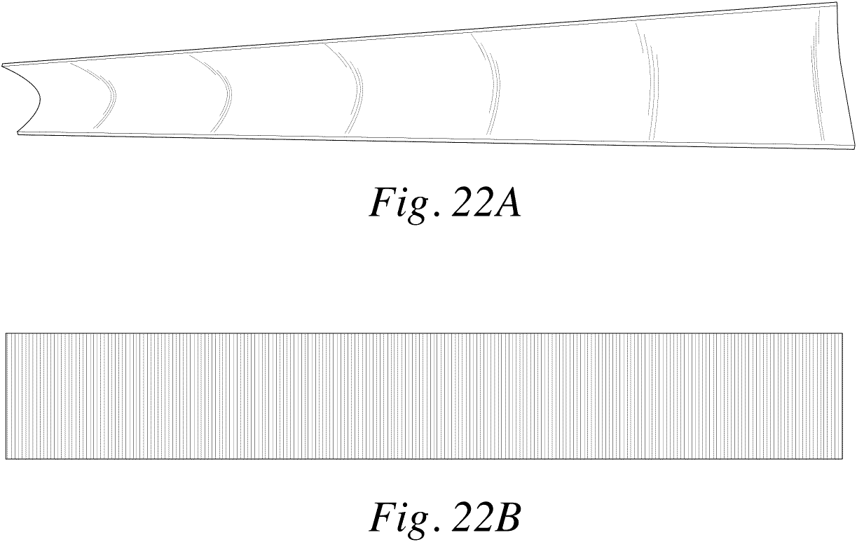

FIGS. 22A-B show perspective and top views of an anisotropic reflector used in the measured plot of 3D measured UV energy shown in FIG. 1. The anisotropic parabolic reflector configuration has diffuse channels perpendicular to the longitudinal reflector. The anisotropic reflector provides a programmable or selectable ratio and area with respect to spectral and diffuse areas. Modifying the width, depth, and spacing of the channels perpendicular to the light path changes the reflector properties and can be designed to address different applications.

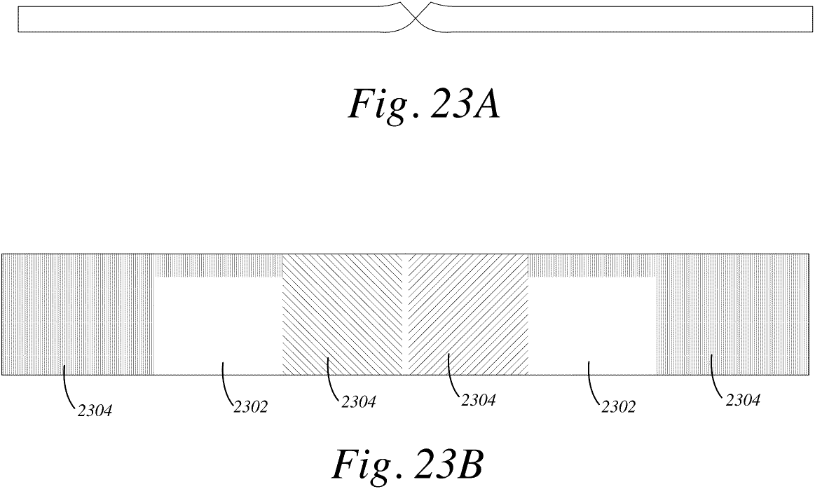

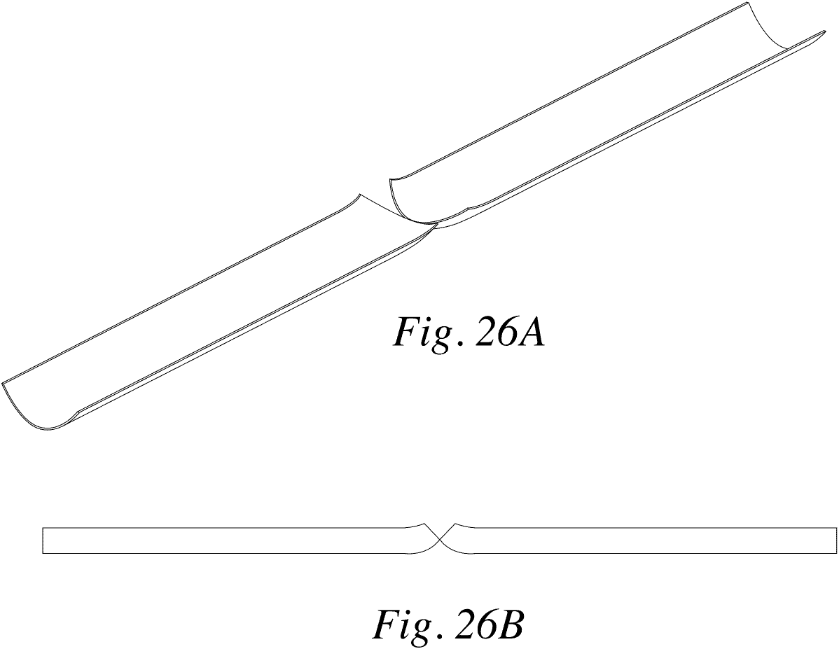

FIGS. 23A-B show an example of two reflectors positioned end to end with each reflector having three different reflector portions. Each reflector includes two different types of anisotropic reflectors combined with a specular reflector. By combining different reflector types into a single device, energy can be distributed uniformly from multiple or a single light source to complex shapes that would traditionally require multiple light sources/optics. The segmented ratio of diffuse, spectral, orientation shape can create a 3D segmented anisotropic reflector. In the depicted embodiment there are portions that are pure spectral 2302 and portions that include a ratio and orientation of spectral and diffuse properties 2304. FIGS. 26A-B show a parabolic reflector with an angled segment to cast energy back into the field in a specific direction.

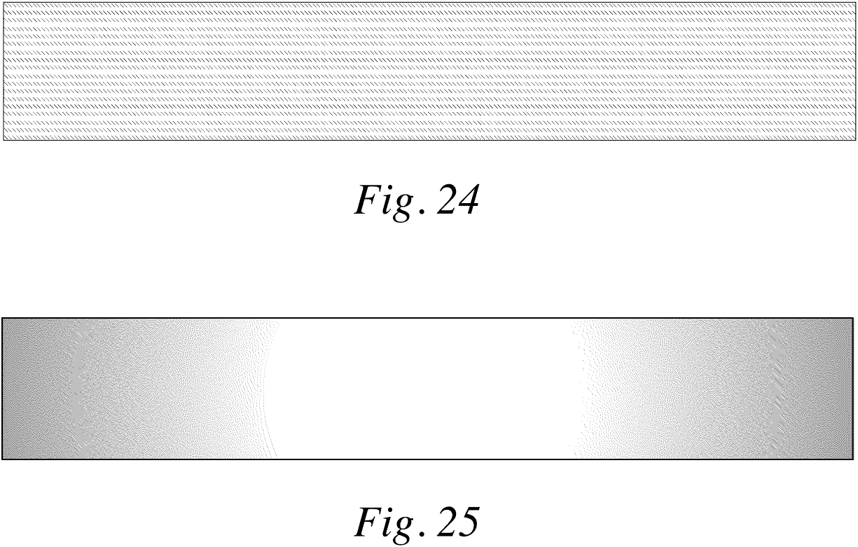

FIGS. 24 and 25 show additional exemplary parabolic anisotropic reflectors. The FIG. 24 reflector has a parallel grain to the parabolic reflector length and the FIG. 25 reflector has a radial pattern of varied grains to produce a desired energy delivery pattern. For example, in FIG. 25 a more spectral pattern is provided toward the center portion of the anisotropic reflector, while a more diffuse pattern is provided at the ends of the anisotropic reflector.

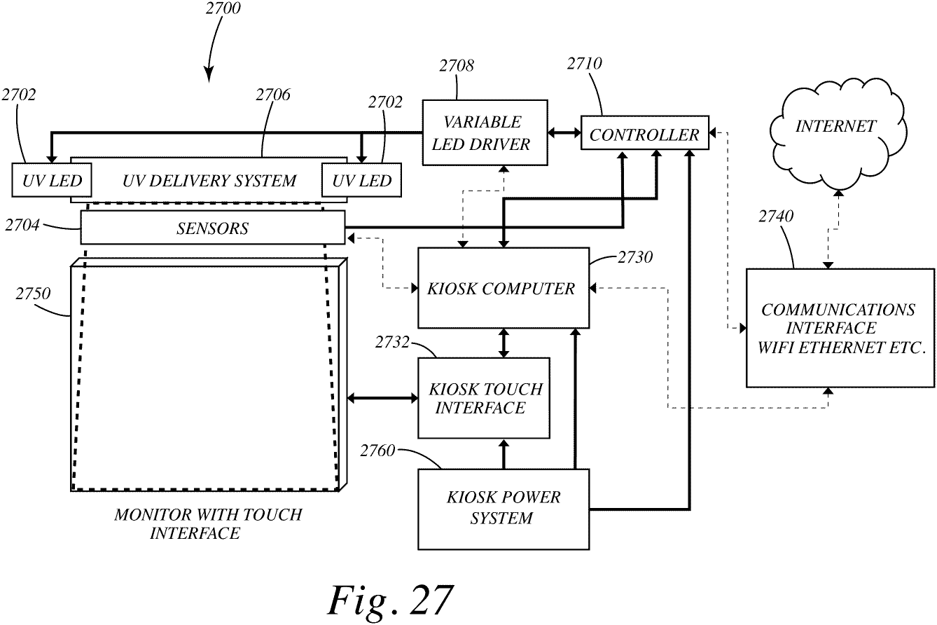

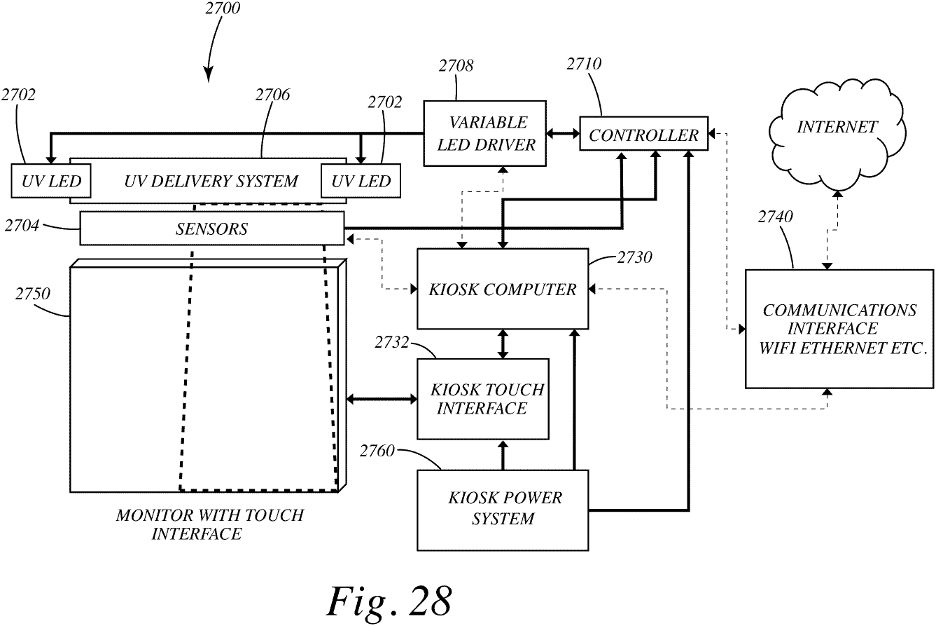

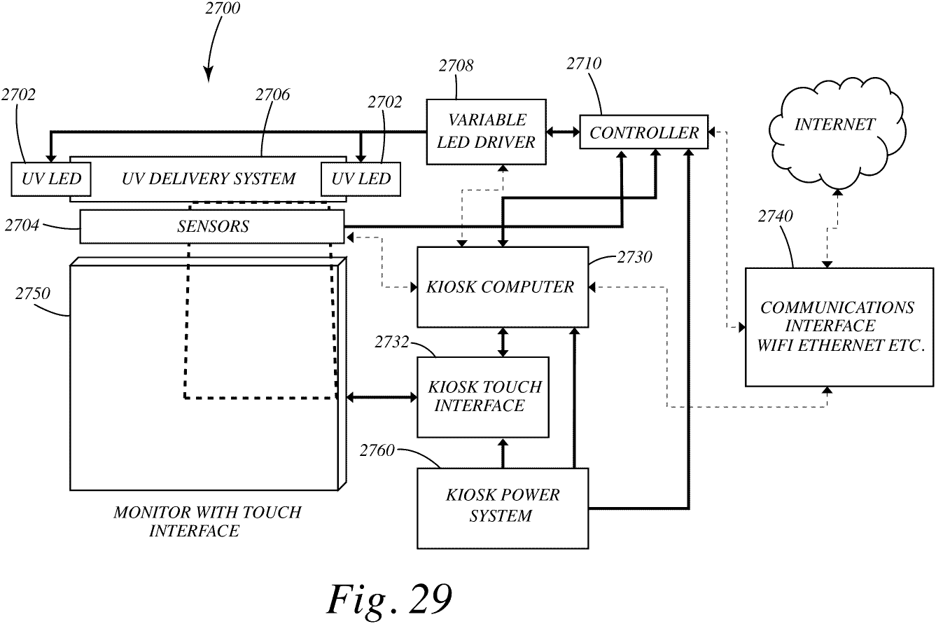

FIG. 27 illustrates an exemplary block diagram of a low profile UV delivery system. It shows the integration of a low-profile energy delivery system 2700 of the current disclosure with a computer 2730 and touch interface 2732 for treating a display 2750. The UV LEDs 2702, sensors 2704, anisotropic channel 2706, variable LED driver 2708, controller 2710 can all be included in the low profile UV delivery system 2700. A communication interface 2740 can be provided to the kiosk computer and low-profile energy delivery system to permit communication to each other or to the Internet. The touch interface can start the timer to validate the touch for treatment and communicate with the system 2700 to control operation of the same. The controller or the kiosk computer can trigger the dose, dose area and duration for the desired area and timing. Sensors, variable LED driver and other components of the system 2700 can receive instructions or other information from the kiosk computer, or touch interface. The controller or kiosk computer can track hours, touches, full treatments, partial treatments, and other UV treatment statistics, which can be uploaded to a server in the cloud for further analysis and presentation to a user. The controller can also be configured to respond to sensors 2704 for proximity based shutoff, movement shutoff, and/or maintenance shutoff. The LEDs can be easily replaced. The cloud interface can provide end of life tracking as well as locally to the controller or kiosk computer depending on control and configuration options. The communications module can have WiFi, Ethernet, Mesh, 3G 5G, or other IoT communications for monitoring functions and performance. The screen treatment shows two LEDs at full power to treat the full screen, but additional or fewer LEDs and anisotropic channel configurations can be utilized in alternative embodiments. The kiosk power system 2760 can be utilized to power the kiosk computer, kiosk touch interface, and the low profile UV delivery system 2700. In some configurations power can be provided in a pass-through configuration. FIG. 28 shows the same system as FIG. 27 but with the right LED on 100% treating the full right side. This illustrates that the low profile UV delivery system can react to touch interface sensor information to treat a targeted area of the display. This can be useful in a number of different scenarios depending on the amount of time available to treat a display, the amount of interaction with the display, and the total amount of desired power output. FIG. 29 shows the same system, but with the right LED on 50% power treating the upper right side. Quadrants can be easily configured and changed programmatically to enable the control to change power for enabling longer LED life.

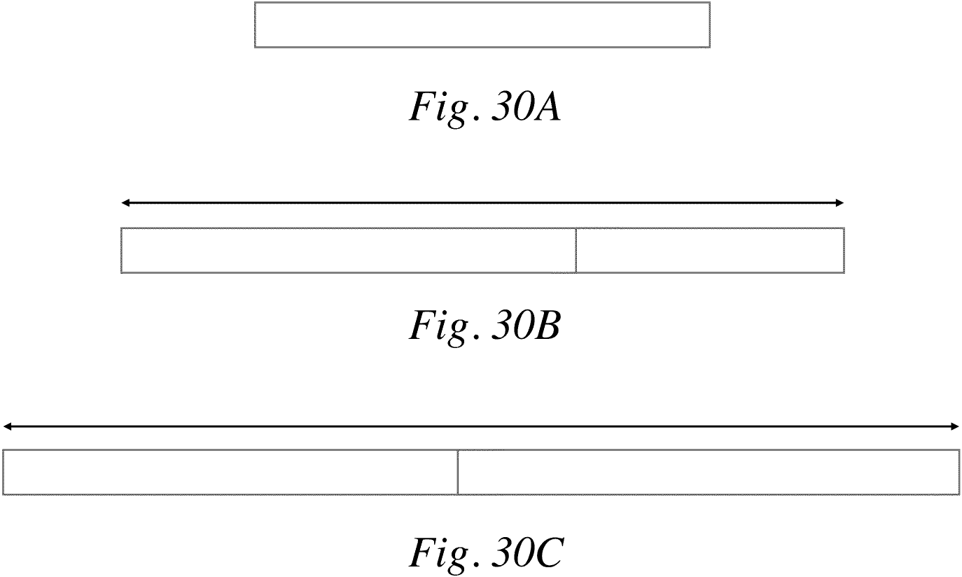

FIGS. 30A-C shows an example of a telescoping treatment unit that can expand to different sizes to treat different display area sizes.

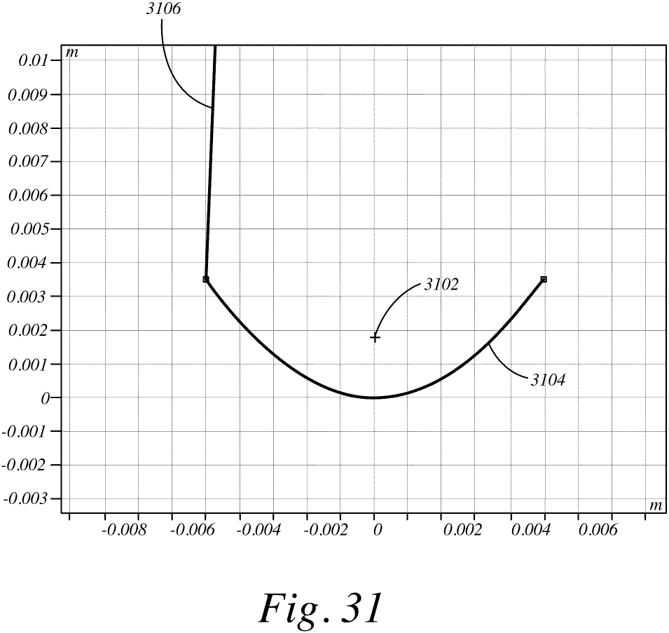

FIG. 31 shows the target focal point on a parabolic reflector for a 10 degree beam angle LED. The LED 3102 is located at the focal point of the reflector 3104. The reflector reflects light toward the display 3106.

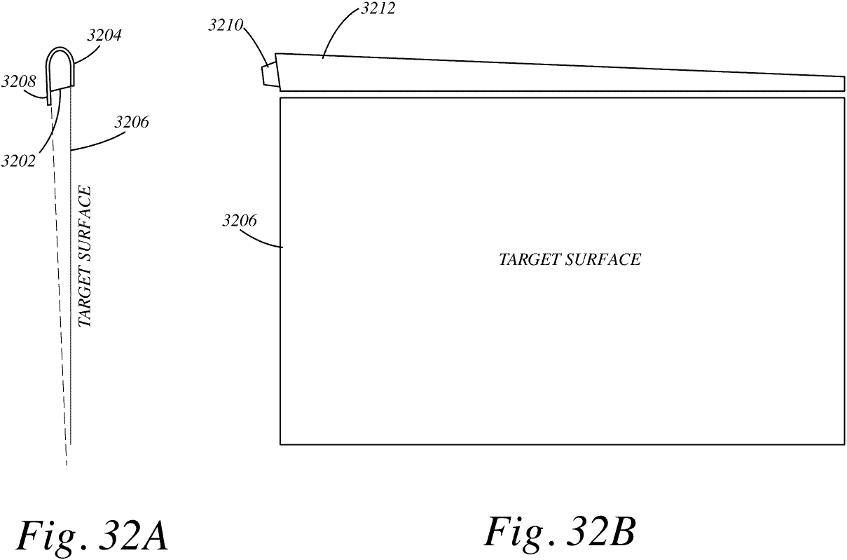

FIGS. 32A-B shows top and end views of one embodiment of a low profile UV delivery system. These figures depict how the low-profile delivery can be implemented using a fused quartz light pipe along with a reflector system and polished surfaces utilizing the distributed light channel method. Referring to FIG. 32A, a lens is set to a display delivery angle 3202 to delivery UV energy to the display 3206. The parabolic reflector 3204 can be provided with an eyebrow 3208. Referring to FIG. 32B, the LED can be set to an entry delivery angle, for example with a lens. The reflector provides a multi-effect reflector surface using a fused quartz light pipe 3212 reflect light toward the target surface 3206.

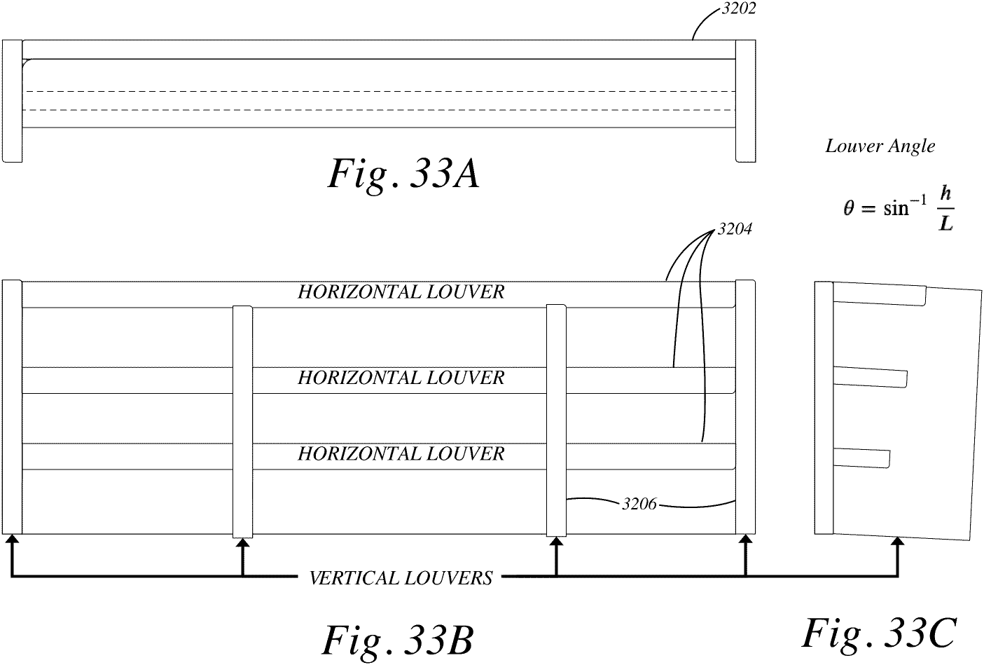

FIGS. 33A-C show one embodiment of a low profile UV delivery system with a UV transmissive window 3302. The UV transmissive window 3302 includes horizontal louvers 3304 and vertical louvers 3306 in their respective axes to limit unwanted UV patterns outside the desired pathing. The louvers allow the energy to be delivered within the specific area and limits exposure at other angles of unwanted interest. FIG. 33A shows a top view, FIG. 33B shows a front view, and FIG. 33C shows a side view.

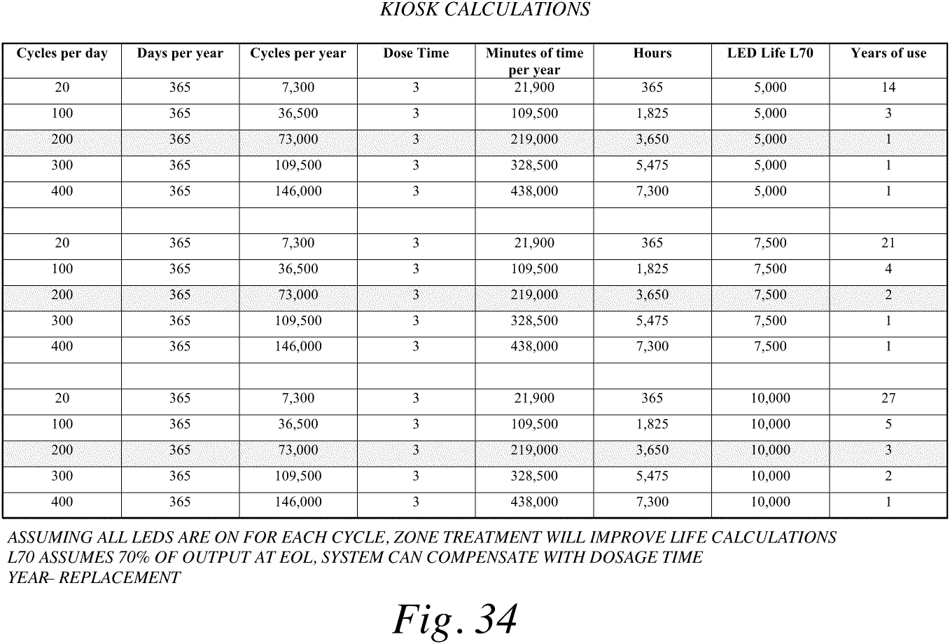

FIG. 34 shows a table of calculations for on time and LED life in one embodiment. L70 is when the LED reaches 70% of life. Although the LED PCBA can be replaceable for easy maintenance, the power and cycle time can utilize present available life times of the UV LEDs. This lifetime is presently increasing every year but infrastructure displays in vehicles for example will last 10 years plus.

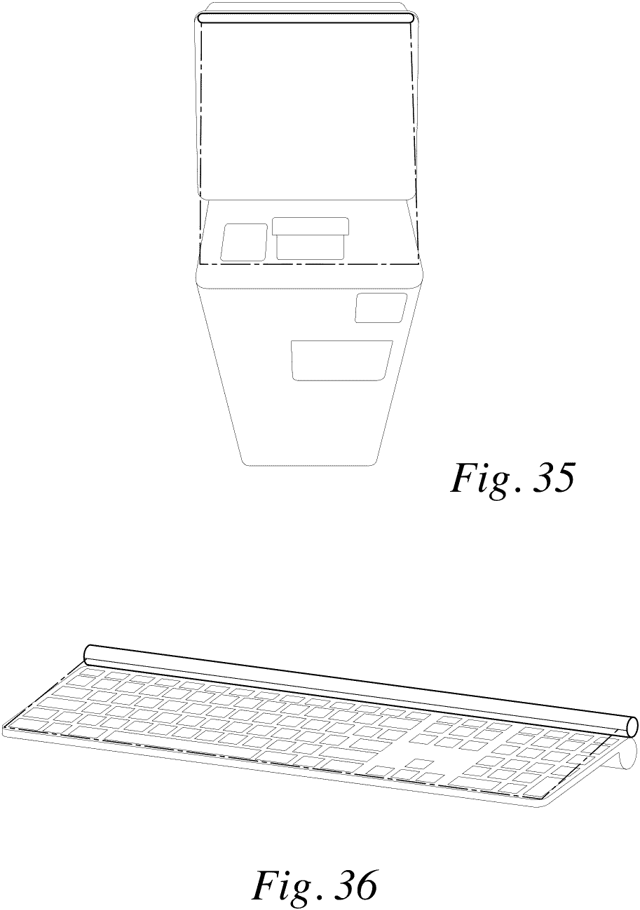

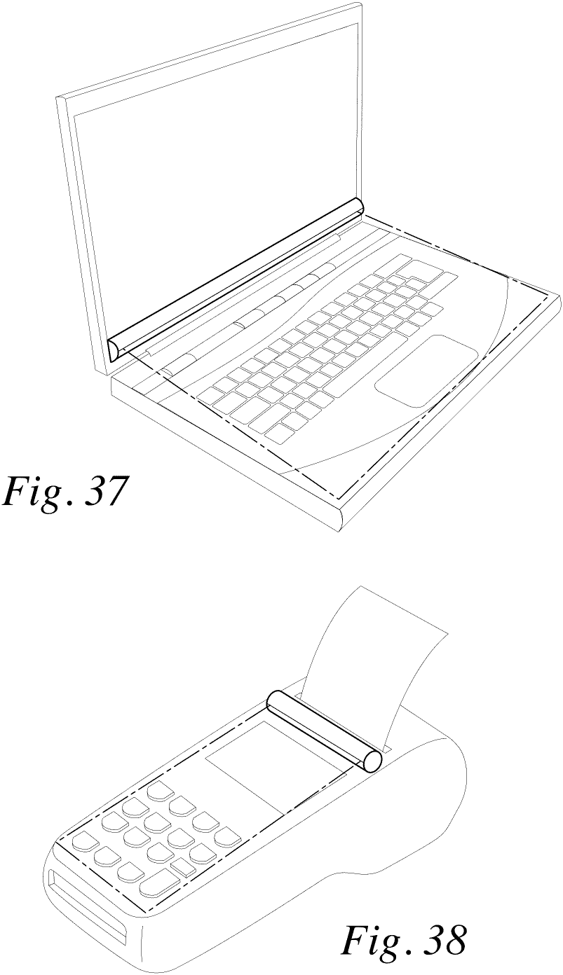

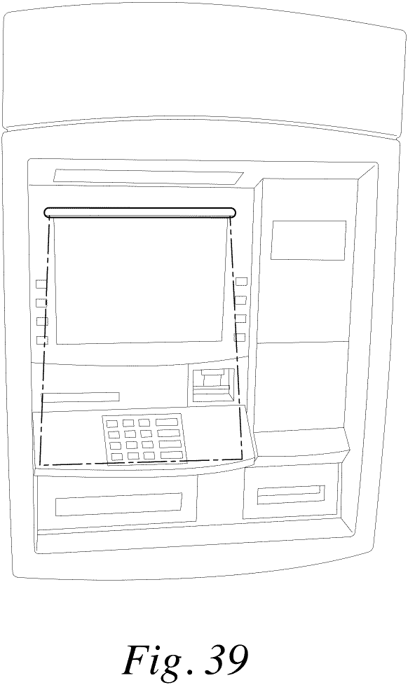

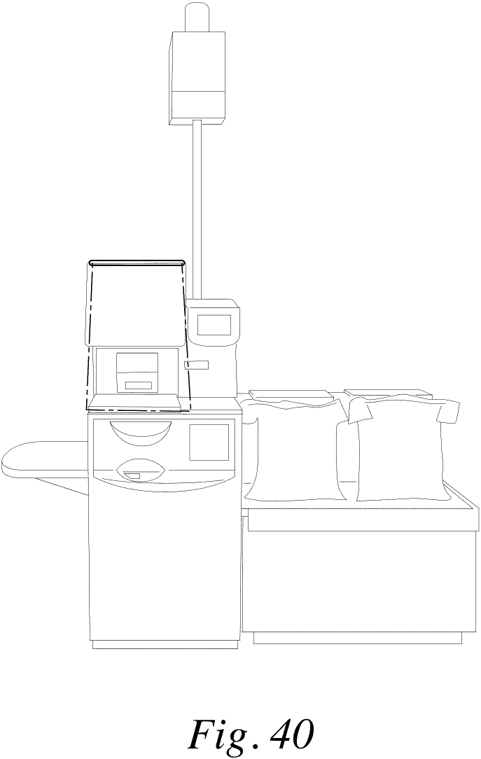

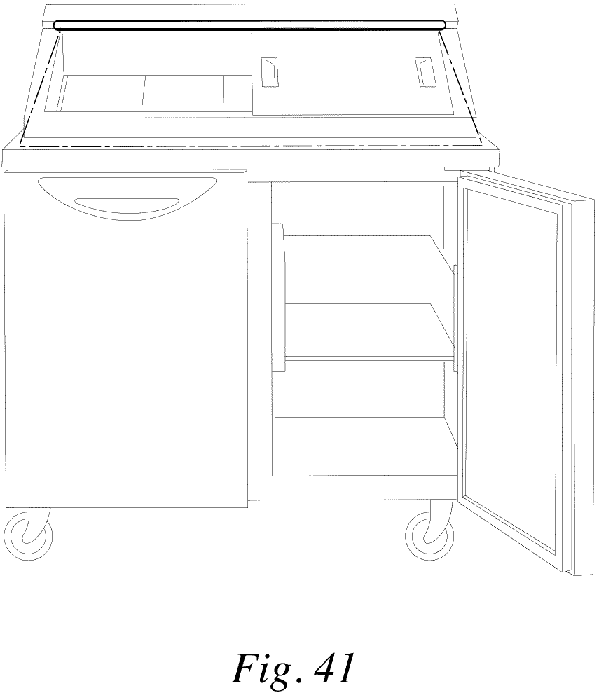

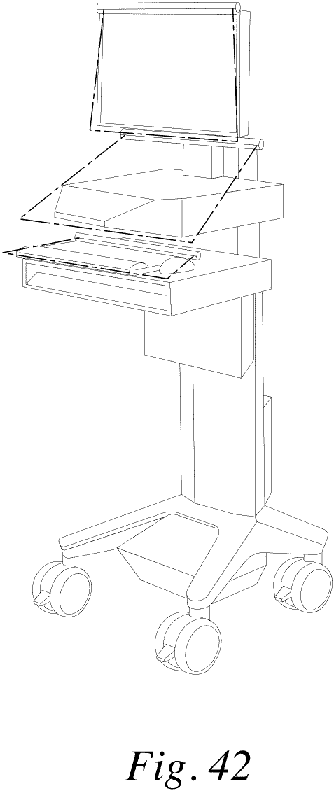

FIGS. 35-42 shows various embodiments of low profile UV delivery systems for different applications. FIG. 35 shows an integrated UV channel system within an airport check in and ticketing kiosk to disinfect high traffic surfaces. FIG. 36 shows a representation of an integrated UV channel system utilized within a keyboard to treat the surface using the kiosk-like control and treatment system. FIG. 37 shows a representation of an integrated UV channel system in a laptop display to disinfect the keyboard surface when the display is at specific angles that can treat or dose the keyboard properly. FIG. 38 shows a representation of an integrated UV channel system within a portable point of sale system to treat the high traffic surface. FIG. 39 shows a representation of an integrated waterproof UV channel system within an ATM to disinfect those high traffic surfaces. FIG. 40 shows a representation of an integrated UV channel system within a retail POS system to treat those high traffic surfaces. FIG. 41 shows a representation of an integrated UV channel system within a food preparation table to disinfect those high traffic surfaces. FIG. 42 shows a representation of an integrated UV channel system within a medical cart treating the monitor, the keyboard and the work surface to disinfect those high traffic surfaces. The low profile nature allows treatment in small spaces like keyboard storage areas.

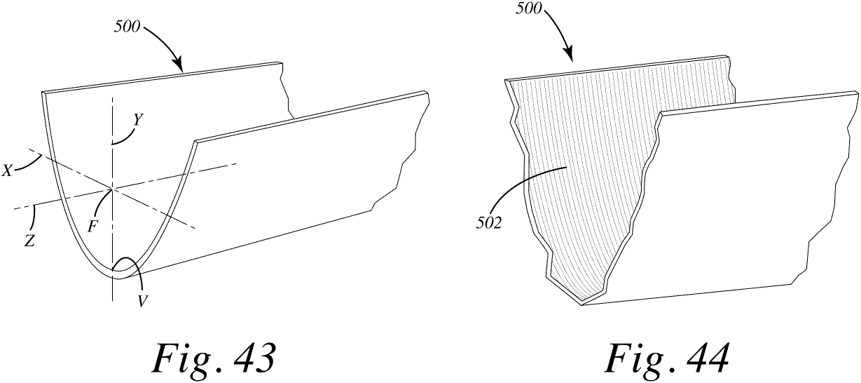

In another alternative aspect, the present invention provides a lighting system for distributing visible light through the use of an anisotropic reflector (See FIGS. 43-55). Generally, the present invention is well-suited for use in a wide range of applications in which it is desirable to distribute light in a programmatic manner over a surface. Although the present invention is described primarily in the context of systems in which uniform light distribution over the surface is desired, the present invention may be tailored to provide illumination that is not uniform. For example, the present invention may be configured to provide regions of greater intensity and lesser intensity in applications where non-uniform distribution is desired.

For purposes of disclosure, this aspect of the present invention will be described in the context of illumination systems for liquid crystal displays (“LCDs”). The present invention is not limited to LCD illumination systems, but may be incorporated into a wide range of applications in which it is desirable to distribute visible light over a surface. Liquid crystal displays are a common form of screen display that often relies on additional illumination to see the screen because the LCDs do not produce light on their own. LCD illumination can be implemented either using frontlighting or backlighting, which refer to the directions the light comes from to illuminate the screen.

The present invention provides channel lighting systems that have the potential to serve as a useful means of diffusing light evenly over the LCD in both frontlighting and backlighting applications. As used herein, the term “channel lighting” refers to illumination using one or more small light sources (such as, but not limited to, a single LED) and a reflector. In typical applications, the present invention may include essentially any type of light source, such as LEDs, incandescent lamps, fluorescent lamps and electric discharge lamps. For example, light sources that have a diameter or width in the range of approximately 1/50^{th} to ⅒^{th} of the distance between the vertex and the focus have proven suitable for many applications. Although typical applications will include a light source that is small relative to the size of the reflector, the size may vary from application to application. Larger light sources may prove to be less efficient, but may prove suitable.

Referring now to FIG. 43, the reflector 500 is in the shape of a parabola that is extended for some length, which may vary from application to application. For example, the reflector may be extended to generally coincide with the length of the object upon which light is to be distributed. For purposes of this disclosure and with reference to FIG. 43, the extrusion direction be referred to as the “Z-direction” Z, and the direction from the vertex V of the parabola to the focal point F of the parabola be the “Y-direction” Y. The X-direction X can be determined using the right-hand rule (i.e., perpendicular to the Z and Y directions). The light source is located at or close to the focal point of the parabola in the XY-plane and directed in the Z-direction, or in some direction that is a combination of the Z-direction and the negative Y-direction. Typically, the light source will be at the focal point, but some level of deviation from the focal point may be acceptable is some application provided that the corresponding reduction in efficiency is acceptable.

Because the light source is small and located at or close to the focal point of the parabolic reflector in the XY-plane, the majority of light from the light source that reflects off the reflector will be collimated in the XY-plane or in the positive Y-direction when viewed in the XY-plane.

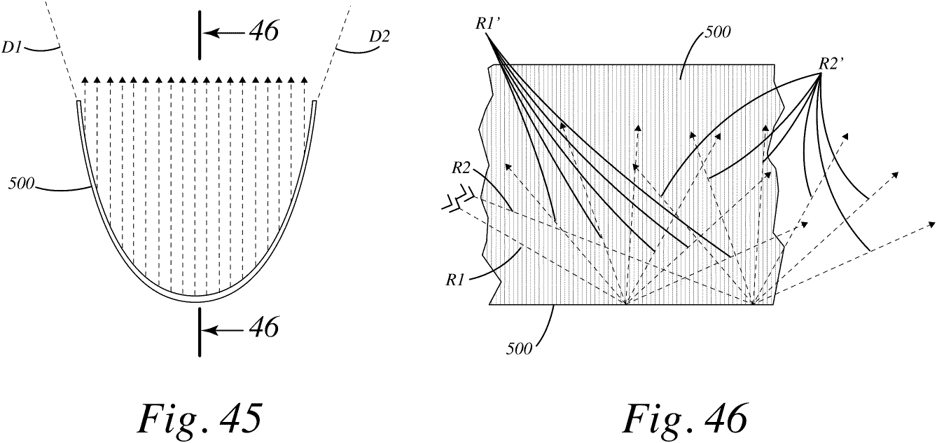

In the illustrated embodiments, the parabolic reflector 500 is an anisotropic reflector, meaning that the reflector is configured to provide different reflective properties in different directions. More specifically, the parabolic reflector 500 of FIG. 44 provides scattering of light in the YZ-plane, without materially scattering the light in other directions. FIGS. 45 and 46 are representative drawings that assist in understanding the anisotropic nature of the parabolic reflector 500. FIG. 45 is a representational sectional view taken transversely across an exemplary parabolic reflector. The arrows in FIG. 45 represent rays of light reflecting from the surface of reflector 500. As can be seen, the rays of light are collimated from the parabolic surface when viewed from this perspective. FIG. 45 shows an idealized representation of the collimation. The actual level of collimation in the XY-plane may vary from application to application depending, for example, on practical limitations and/or on intentional design choices that may be made to achieve a desired light distribution profile. In some applications, light may leave the mouth of the anisotropic reflector with a divergence in the XY-plane of up to about 30 degrees full width at half maximum as illustrated with reference to lines D1 and D2 in FIG. 45. In FIG. 45, line D1 extends at an angle of about +15 degrees relative to the axis of symmetry of the parabola defined by the cross-sectional shape of the anisotropic reflector. Similarly, line D2, extends at an angle of about -15 degrees relative to the axis of symmetry. In the illustrated example, lines D1 and D2 represent the lines along which the light intensity is reduced to about one-half the maximum light intensity. More specifically, the intensity of light measured at any point along the mouth of the reflector between lines D1 and D2 is greater than one-half the maximum intensity and the intensity of light measured outside the region between lines D1 and D2 (i.e., to the left of line D1 and to the right of line D2) is less than one-half the maximum intensity (subject to aberrations in the light source). In alternative applications, the acceptable level of divergence in the XY-plane may vary. For example, in alternative applications, the anisotropic reflector may have an acceptable divergence in the XY-plane of up to about 25 degrees full width at half maximum, up to about 20 degrees full width at half maximum, up to about 15 degrees full width at half maximum, up to about 10 degrees full width at half maximum, up to about 5 degrees full width at half maximum, up to about 3 degrees full width at half maximum, up to about 2 degrees full width at half maximum, up to about 1 degree full width at half maximum, or about 0 degree full width at half maximum.

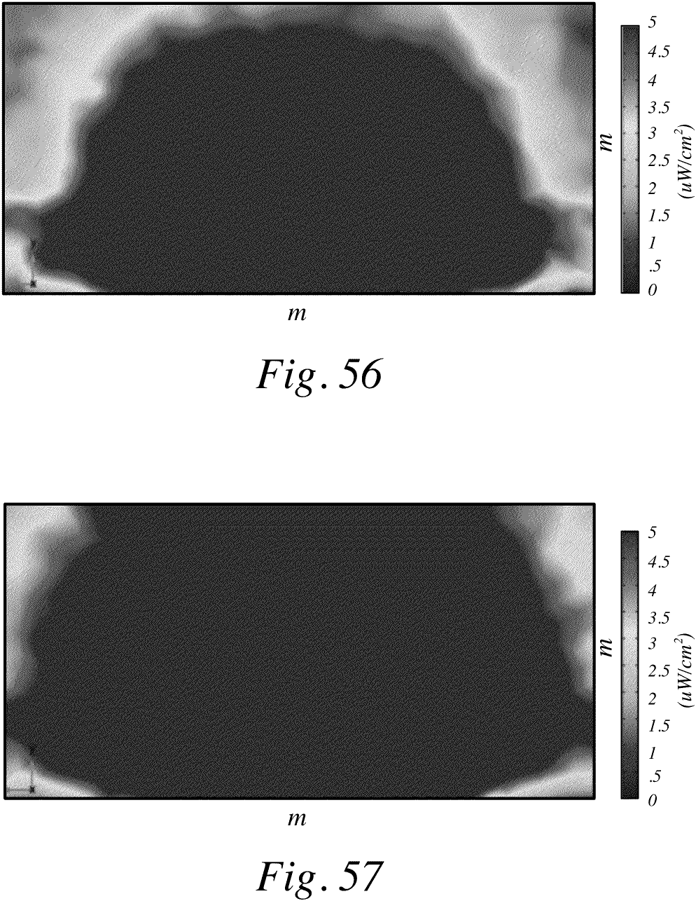

FIGS. 56 and 57 illustrate exemplary UV treatment patterns generated by a beam divergence of 13.7 degrees half-angle and 6.1 degrees half-angle, respectively. The parabolic reflector does not collimate the light perfectly in the X-Y plane. Instead, some amount of beam divergence remains. In general, as beam divergence increases, the light becomes less uniformly distributed, limiting the size of target that can be illuminated sufficiently with a particular configuration. Accordingly, the configuration and specific parameters of a low profile UV light delivery system can be adjusted to suit a particular application.

The amount of beam divergence can be selected as a function of the size of the light source and the curvature (second derivative) of the parabola that defines the anisotropic reflector. Accordingly, the beam divergence can be configured to a suitable angle by selecting the light source and the curvature of the anisotropic reflector. Beam divergence is positively correlated with both of these factors, meaning that lower divergence angles occur for small light sources and low-curvature parabolas, and higher divergence angles for the inverse. Since high-curvature parabolas generally do not need to be as tall to capture the same amount of light from a given source, there is a tradeoff between the uniformity of reflected light (as well as how big of a target can be illuminated) vs. the height of the reflector. Reflector-light source combinations can be selected to provide a low profile that will perform suitably for a given application.

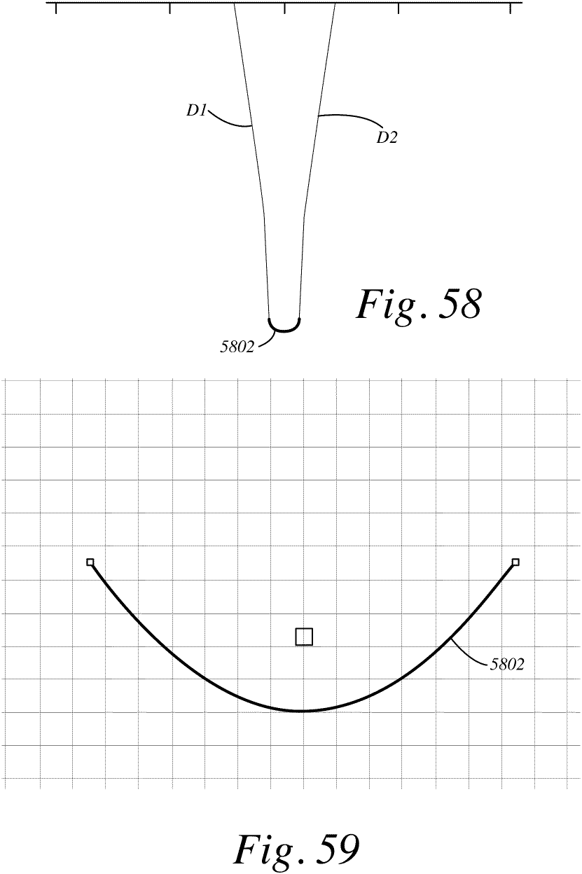

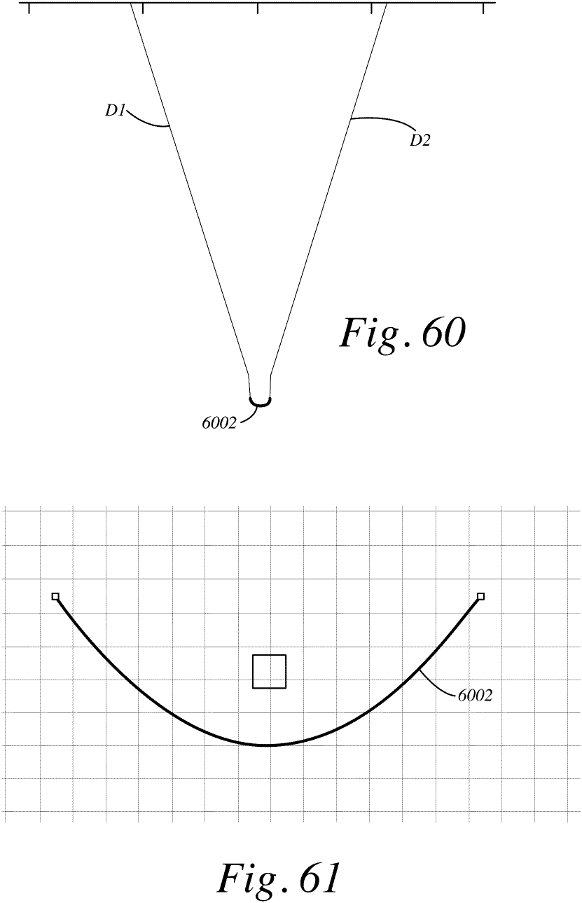

FIGS. 58-61 further illustrate beam divergence and its effects within various embodiments of low profile UV delivery systems. In particular, these figures show how the relationship between size of the LED die and the height of the reflector influences beam divergence. FIGS. 58-59 illustrate an exemplary 1″ parabolic reflector 5802 in conjunction with a 1 mm x 1 mm LED die. The resulting beam divergence (Di, D₂) of the reflected light is 9.5 degrees full width at half maximum. FIGS. 60-61 illustrate a 0.5” reflector with a 1 mm x 1 mm LED die that results in a beam divergence (Di, D₂) of reflected light that is 15.2 degrees full width at half maximum. These figures illustrate that altering the height of the parabolic reflector alters the reflected light beam divergence. When combined with other factors, such as the angle of the reflector relative to the screen, a suitable configuration can be provided to cast reflected light over a particular screen size. Other configurations can adjust other parameters such as the LED die size or beam entry angle of the LED relative to the parabolic reflector to achieve suitable coverage of a particular size display.

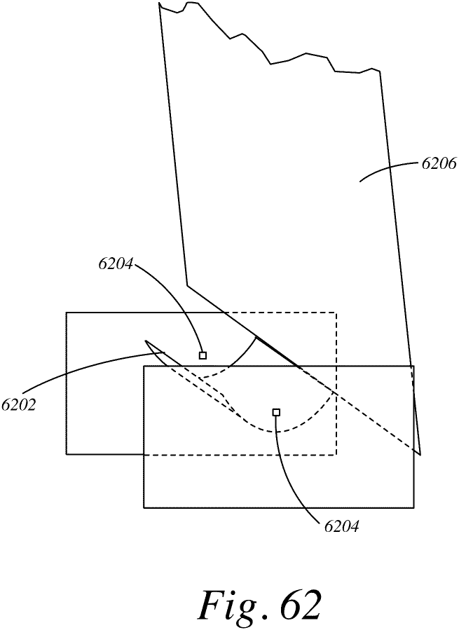

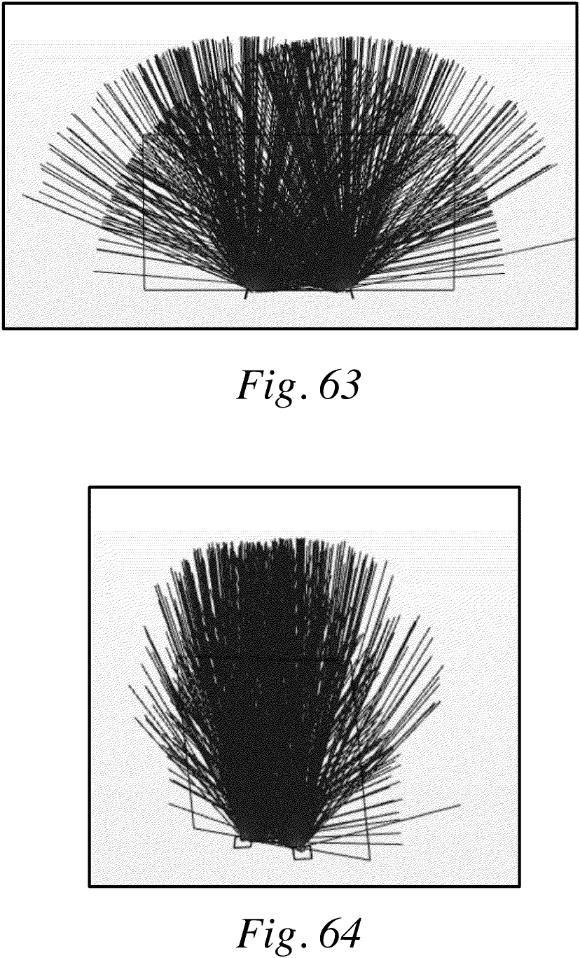

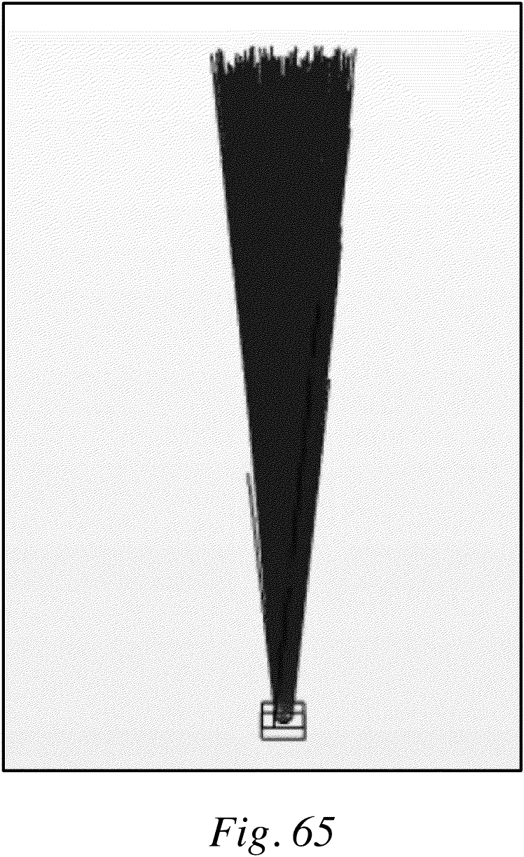

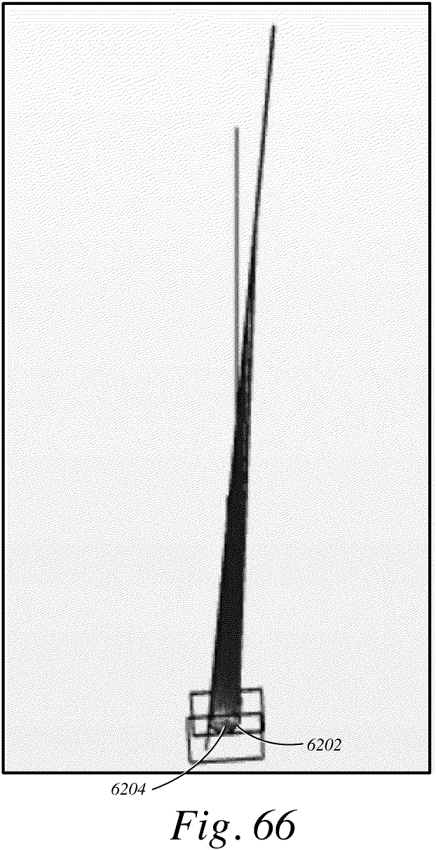

FIG. 62 illustrates a parabolic reflector 6202 with LEDs 6204 on both ends, and a target screen 6206 adjacent. FIG. 62 utilizes the exemplary 1″ parabolic reflector of FIGS. 58-59 with a 1 mm LED die. FIGS. 63-66 illustrate various views of light projection from the parabolic reflector. FIG. 63 illustrates a YZ-view that shows how UV light is scattered after reflection to provide a UV energy distribution across the display 6206. FIG. 64 provides an isometric view. FIG. 65 illustrates an X-Y view that shows light is generally collimated, with a beam divergence of 9.5 degrees full width at half maximum. FIG. 66 shows another XY-view that emphasizes a small angle of incidence at which the UV light strikes a target surface.

Accordingly, there are a number of selectable parameters of a low profile UV delivery system to provide suitable treatment. Such parameters can include the height, orientation (e.g., beam divergence with respect to the target display), material, and position of the anisotropic reflector as well as the relative position, size, power level, and orientation (e.g., beam entry angle) of the LED with respect to the parabolic reflector.

FIG. 46 is a representational sectional view of the parabolic reflector 500 taken along line 46-46 in FIG. 46. The arrows represent the potential paths of two rays of light, R1 and R2, that are emitted by a light source and reflect from the parabolic reflector. As can be seen, each ray of light, R1 and R2, may reflect from the reflector at a range of different angles, R1′ and R2′, depending on the surface texture on the parabolic reflector 500 at the point of reflection. As a result, even though this light remains essentially collimated in the XY-plane, it is still propagating with a non-zero Z-component (i.e., the light is scattered in the YZ-plane). FIG. 46 illustrates a representative scattering in the YZ-plane. The actual degree of scattering in the YZ-plane may vary from application to application depending, for example, on practical limitations and/or on intentional design choices that may be made to achieve a desired light distribution profile. In some applications, light reflecting from the surface of the anisotropic reflector will have scatter light of have a divergence in the YZ-plane of about 120 degrees full width at half maximum. In alternative applications, the desired level of divergence in the YZ-plane may vary. For example, in alternative applications, the anisotropic reflector may diverge in the YZ-plane at least about 100 degrees full width at half maximum, at least about 90 degrees full width at half maximum, at least about 75 degrees full width at half maximum, at least about 60 degrees full width at half maximum, at least about 45 degrees full width at half maximum, at least about 30 degrees full width at half maximum, or at least about 15 degrees full width at half maximum.

In some applications, it may be desirable to vary the degree of divergence over different regions of the anisotropic reflector. For example, it may in some applications be desirable to have maximum divergence (e.g., 120 degrees full width at half maximum) in the YZ-plane toward the middle of length of the reflector and to progressively reduce the degree of divergence in the YZ-plane toward opposite ends of the reflector. This may, in some applications, help to reduce the amount of light that is reflected beyond the edges of the target surface. In some applications, the divergence of reflecting light at any given point along the reflector will be set to provide the desired distribution of light over the target surface while reducing the amount of light that may be lost by virtue of being reflected to a location off of the target surface.

The parabolic reflector may be configured to provide YZ-plane scattering in essentially any way, such as through the use of surface treatments, surface coatings or through variations in surface shape. For example, in the embodiment shown in FIGS. 44 and 46, the parabolic reflector 500 has a surface finish 502 consisting of relatively small grooves that have a generally constant Z-position. The surface texturing 502 shown in FIGS. 44 and 46 is intended to be representative and not a precise reproduction. The number, size, shape, density, depth and other characteristics of the surface texture may vary from application to application to provide the desired level of scattering in the YZ-plane. As shown representatively, each individual groove extends transversely (or laterally) across the surface of the reflector in essentially the x-direction. In one embodiment, the surface of the reflector is treated with sandpaper, such as 220 grit sandpaper, to create a surface treatment including a plurality of small, parallel grooves (or scratches) that extend transversely across the reflector surface along the X-direction (e.g., extending generally perpendicular to the Z-direction). The number and/or depth of grooves (or scratches) may vary from application to application. As a result of the transverse grooves, the reflection of a ray (e.g., R1 or R2) in the XY-plane is governed by the shape of the parabola, and thus collimation in the XY-plane is maintained despite the fact that the reflection can change the Z-component of propagation. The net result of this optical arrangement is that light scatters or spreads from the point of reflection in, for example, a roughly triangular fashion in the YZ-plane, while remaining generally collimated in the XY-plane. Since the light rays R1 and R2 intersect the reflector 500 at sundry Z-positions, the final pattern of light reflected by the reflector 500 is roughly trapezoidal in the YZ-plane while remaining collimated in the XY-plane.

When light from the reflector 500 is projected onto a screen (or other surface to be illuminated) that lies at an angle to the parabola, this results in a highly uniform distribution of optical energy across the screen (or other surface). This is because gradients in light intensity are small in the X-direction (zero in the idealized case), and the projection of the screen perpendicular to ray propagation lies almost entirely in the X-direction when viewed from the XY-plane.

This parabolic reflector with grooves that have constant z-position is referred to herein as one example of an “anisotropic reflector,” because it has reflection properties that are different depending on the plane in which the rays are viewed. As discussed above, viewed in the XY-plane, the reflector 500 behaves very similarly to a specular reflector (See FIG. 45), while viewed in the YZ-plane the reflector 500 behaves similarly to a diffuse reflector (See FIG. 46). In the illustrated embodiment, the light, when viewed in the XY-plane, is generally collimated and, when viewed in the YZ-plane, is generally scattered. As discussed elsewhere herein, the level of collimation in the XY-plane (e.g., acceptable level of divergence of light in the XY-plane) and the characteristics of scattering in the YZ-plane (e.g., range of angles over which the light is scattered and the distribution profile of light over that range) may vary from application to application. Further, the level of collimation in the XY-plane and the characteristics of the scattering in the YZ-plane may vary through different portions of the anisotropic reflector. For example, the anisotropic reflector may have different portions that provide different levels of collimation in the XY-plane and/or different portions that provide different characteristics of the scattering in the YZ-plane.

As described above, the present invention is well-suited for use in LCD illumination systems. Channel lighting embodiments in accordance with the present invention are capable of providing a low-profile source that can be used to deliver a substantially uniform illumination field over a surface, such as, but not limited to, flat surfaces typically included in LCDs. This is useful for a variety of LCD applications including but not limited to frontlighting an LCD display uniformly and backlighting an LCD display uniformly. Although the present invention is described in the context of illuminating flat surfaces, the present invention may be adapted for use in illuminating surfaces that are not flat.

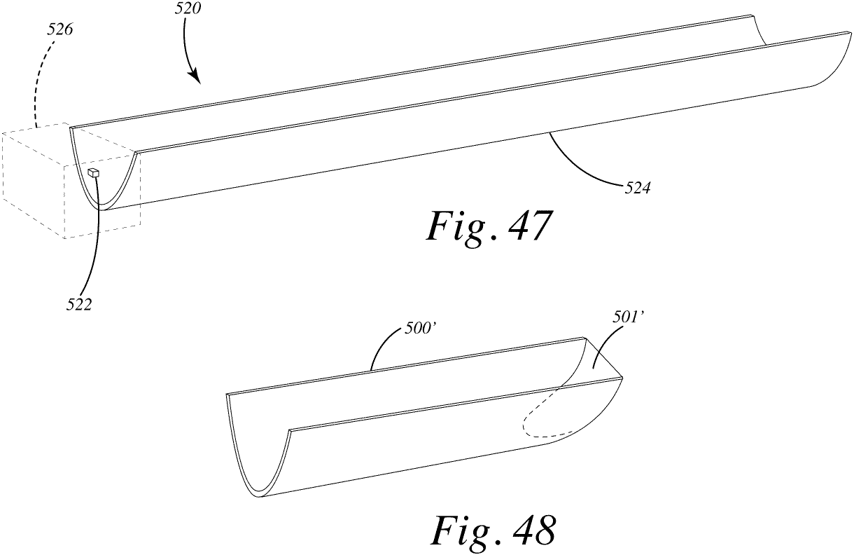

FIG. 47 is a representational view of a channel lighting system 520 in accordance with one embodiment of the present invention. The channel lighting system 520 generally includes a light source 522 and a parabolic reflector 524. The light source 522 is installed within a housing 526 that may contain all associated electronics, including any controller, power supply components and heat sink(s) (not shown). The light source 522 in this embodiment is a single LED, but, as noted above, it may be essentially any type of light source, such as single/multiple LEDs, incandescent lamps, fluorescent lamps and electric discharge lamps. In use, a single channel lighting system 520 may be positioned along one edge of a surface to be illuminated. For example, in one application, a single channel lighting system 520 may be provided with a parabolic reflector 524 of essentially the same length as the edge of the surface along which it is positioned. In other applications, a plurality of channel lighting systems 520 may be provided to cooperatively illuminate a surface. For example, a plurality of channel lighting systems 520 may be positioned along one or more edges of a surface. In one alternative embodiment, two channel lighting systems 520 may be positioned end-to-end along an edge of the surface to be illuminated (e.g., each approximately half the length of the edge). FIG. 48 (described in more detail below) is an example of a reflector 500′ that might be incorporated into a shorter channel lighting system. In another alternative embodiment, two channel lighting systems 520 may be positioned along opposite edges of the surface to be illuminated (each extending approximately the full length of the edge). When a plurality of channel lighting system 520 are provided, they may be configured to be turned on and off simultaneously by a single a controller or by a plurality of coordinated controllers. Alternatively, the different channel lighting systems 520 may be configured to operate independently from one another, which may allow them to be turned on and off simultaneously or separately. This may be used to allow the system to selectively provide different types of light (e.g., UV or visible light or different color visible lights) or different intensity levels (e.g., light from a single channel lighting system or a plurality of channel lighting systems).

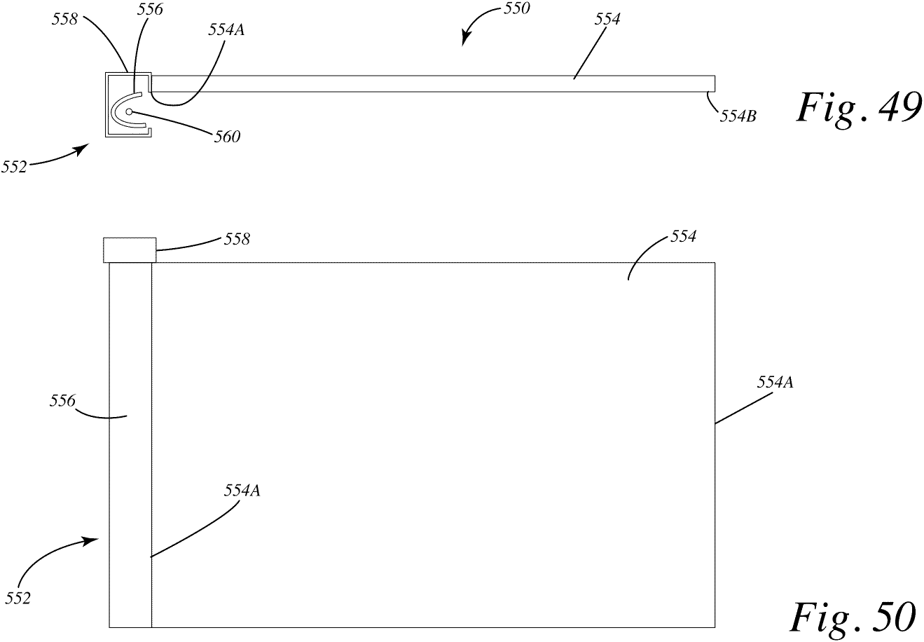

FIGS. 49 and 50 are schematic representations of an LCD illumination system 550 in accordance with an embodiment of the present invention. FIGS. 49 and 50 show a channel lighting system 552 that is configured to distribute light over a surface 554. The channel lighting system 552 includes a parabolic reflector 556 that is oriented at an angle to the surface 554 so that light reflecting off the reflector 556 is directed across the surface 554. The channel lighting system 552 includes a light source 560 (e.g., LED) positioned within a housing 558. Although not shown, the housing 558 may contain the controller, power supply and other electronics that may be needed to operate the light source 560. The parabolic reflector 556 may be configured (e.g., sized, shaped and oriented) so that essentially all of the reflected light is cast upon the surface 554 beginning at the proximal edge 554a of the surface 554 and continuing across the surface 554 to the distal edge 554b. FIGS. 49 and 50 can be seen to be representative of a frontlighting LCD illumination system. In that context, the surface 554 upon which the light is distributed by the channel lighting system 552 is the front surface of an LCD. In frontlighing LCD illumination systems, the channel lighting system 552 may be disposed in a housing (not shown) that extends forward of the front surface of the LCD. Alternatively, FIGS. 49 and 50 can be seen to represent a backlighting LCD illumination system. In that context, the surface 554 upon which the light is distributed by the channeling light system 552 is the back surface of the LCD, where light from a conventional backlighting system would enter the LCD.

Although the illustrated embodiment of FIG. 47 shows a channel lighting system 520 with a single LED 522 located at one end of the parabolic reflector 524, the channel lighting system may include additional LEDs when additional light is desired. For example, in alternative embodiments, one or more additional light sources (e.g., LEDs) may be positioned adjacent to LED 522. As another example, one or more additional light sources may be positioned at the end of the reflector opposite the illustrated LED. As an alternative to using a single reflector that extends the full length of the surface to receive light, the channel lighting system may include a plurality of reflectors that collectively extend along the full length of the surface. For example, the channel lighting system may include two parabolic reflectors that each extend along approximately ½ the length of the surface. As noted above, FIG. 48 shows an exemplary reflector 500′ that may be suitable for use in an end-to-end arrangement. For example, two of these reflectors 500′ may be positioned along the edge with their closed ends 501′ adjacent to one another. In this example, each of the parabolic reflectors 500′ may have its own light source (not shown in FIG. 48).

FIG. 47 shows a reflector 524 that has an open end and is of essentially the same cross-sectional shape along its length. If desired, the end of each reflector opposite its corresponding light source may be shaped to reflect light reaching the end of the reflector toward the surface to be illuminated. For example, the end of the reflector may itself be shaped to provide the desired reflective pattern to light reaching the end of the reflector. The reflector 500′ of FIG. 48 is a representative example of a parabolic reflector with a closed end 501′. The size, shape and configuration of the closed end 501′ of reflector 500′ is merely exemplary. The characteristics of the closed end 501′ may from application to application to provide the desired reflective pattern. As another example, a separate component, such as a separate end reflector (not shown), may be disposed at the far end of the parabolic reflector to redirect light to the surface to be illuminated. The end reflector may be positioned near the end of the parabolic reflector opposite the light source, either inside or outside the parabolic reflector. As another alternative embodiment, the cross-sectional shape of the reflector may vary along its length. For example, in one alternative embodiment (not shown), the parabolic reflector may maintain a parabolic shape with a common focal point, but become progressively smaller toward the end opposite the light source. The variation in size of the reflector may vary from application to application.

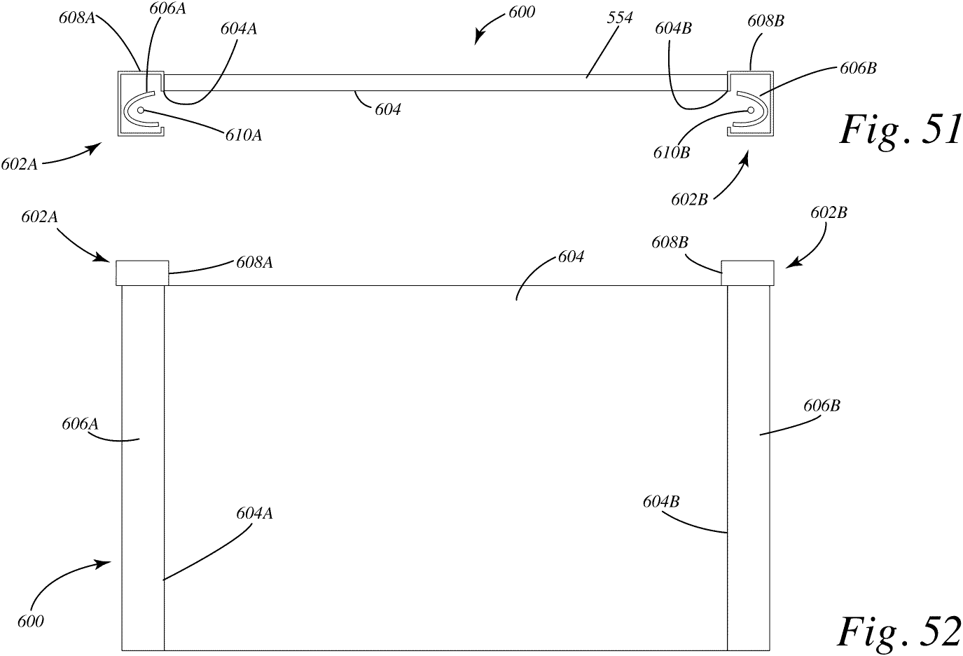

As shown in FIGS. 49 and 50, the LCD illumination system 550 includes a channel lighting system 552 along only one edge of the surface to be illuminated. When desired, additional light sources and corresponding parabolic reflectors can be included along additional edges of the surface to be illuminated. For example, an alternative LCD illumination system 600 is shown in FIGS. 51 and 52. LCD illumination system 600 includes two channel lighting systems 602a and 602b positioned along opposite edges of the surface to be illuminated 604. The two channel lighting systems 602a-b include corresponding light sources 610a-b and reflectors 606a-b. The light sources 610a-b and all associated electronics are positioned in housings 608a-b, respectively. In the illustrated embodiment, each parabolic reflector 606a-b is configured (e.g., sized, shaped and oriented) so that essentially all of the reflected light is cast upon the surface 604 beginning at the proximal edge 604a or 604b and continuing across to the distal edge 604a or 604b. As a result, the two channel lighting systems 602a-b emit essentially overlapping and generally coextensive illumination patterns on the surface 604. In alternative embodiments, the two channel lighting systems 602a-b may be configured to provide different illumination patterns. For example, the two channel lighting system 602a-b may illuminate different portions of the surface 604. To further illustrate, channel lighting system 602a may be configured to illuminate one half of the surface 604, while channel lighting system 602b may be configured to illuminate the other half of the surface 604. In some applications, it may be desirable for one channel lighting system to cast its light on the front surface and the other channel lighting system to cast is light on the rear surface.

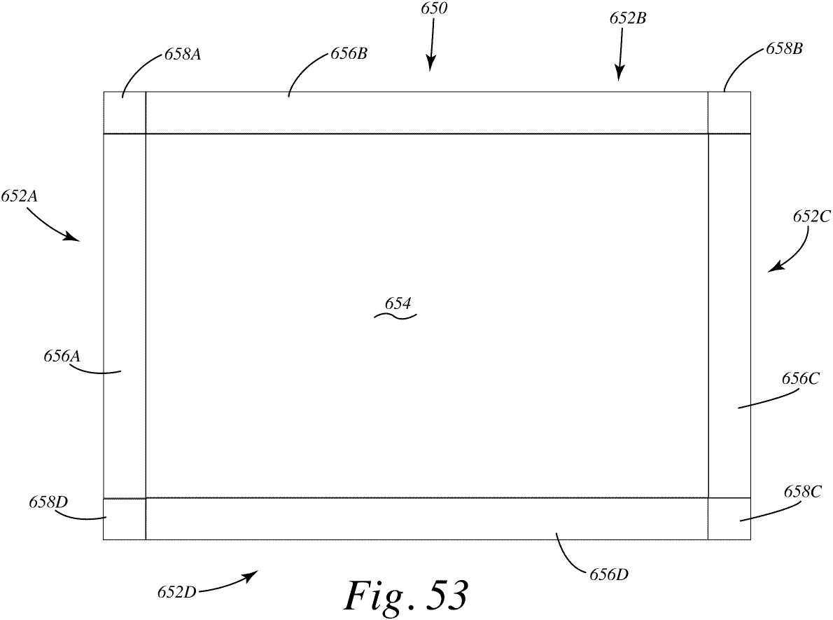

As another example, an alternative channel lighting system may include a separate light source and parabolic reflector along each edge of the surface to be illuminated. In the context of illuminating a rectangular surface, the channel lighting system may include four light sources and four corresponding parabolic reflectors arranged along the four edges of the surface (see FIG. 53). In the embodiment of FIG. 53, the LCD illumination system 650 includes four channel lighting systems 652a-d - one extending along each edge of the surface 654. The channel lighting systems 652a-d of FIG. 53 includes four light housings 658a-d located in the four corners. Each of the light housings 658a-d may include a light source (not shown), such as a single LED that illuminates a corresponding one of the parabolic reflectors 656a-d. Alternatively, each housing 658a-d may include a plurality of light sources that illuminate both adjacent parabolic reflectors 656a-d. For example, housing 658a may include a first LED directed down parabolic reflector 656a and a second LED directed down parabolic reflector 656b. Combing light sources for different reflectors in a single housing can be used to provide light from opposite ends of each reflector. Alternatively, it can be used to allow the LCD illumination system 650 to be implemented with only two housings.

As can be seen, channel lighting can be combined with a traditional diffuser layer that is present in current LCD screens, where the even distribution of light from a single light source or a plurality of light sources arranged along edges of the surface is spread out over the diffuser. As such, the present invention has the potential to reduce the number of light sources that must input their light into the diffuser to achieve uniformity.

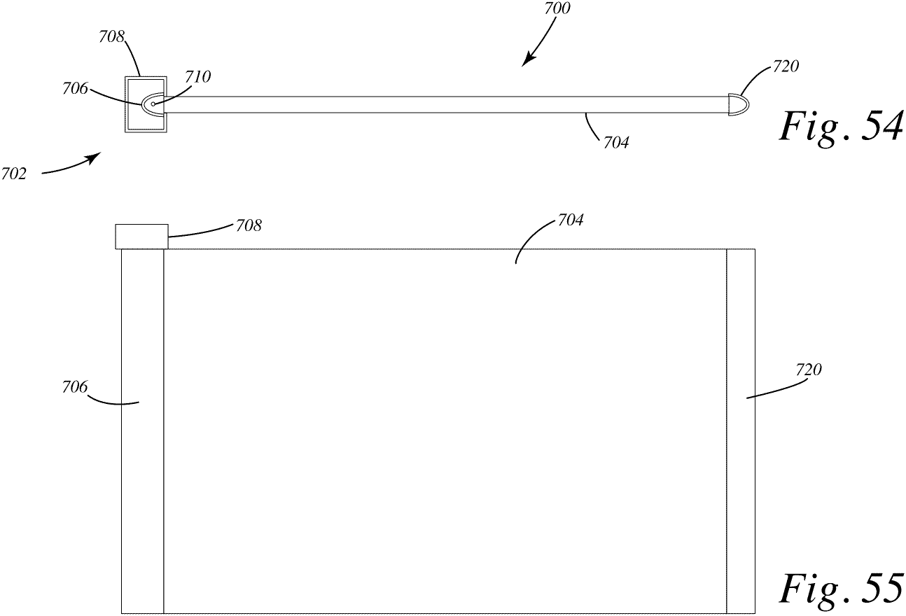

FIGS. 54 and 55 show another alternative embodiment of the present invention in which the LCD illumination system 700 includes a backlight screen diffuser 704 that collects the light from the channel lighting system 702 and distributes it toward the LCD (not shown). In this embodiment, the channel lighting system 702 includes a single light source 710 and parabolic reflector 706 located along one edge of the diffuser 704. A passive reflector 720 may be disposed along the opposite edge of the diffuser 704 to reflect back light that reaches the far end of diffuser 704. The shape of the passive reflector 720 may be selected to provide the desired light distribution. As shown, the passive reflector 720 may be curved, for example, parabolic. As an alternative to the illustrated curved reflector 720, the backlighting application can be combined with a straight mirror on the far side (not shown), angled towards the LCD screen, in order to capture glancing light and redirect it towards the LCD rather than back into the diffuser 704.

The backlight screen diffuser 704 may be provided with the desired light diffusing properties using one or more alternative light diffusing techniques. For example, the light diffusing properties of the backlight screen diffuser 704 can be provided through the use of a lenticular lens (not shown) with physical steps in the geometry, or through the use of a transmissive material with holes laser drilled into the surface with the holes being progressively deeper at greater distances from the parabolic reflector 706 and/or any other system or method for controlling the distribution of light set forth in WO 2021/080638A1, entitled “OPTICAL PROPERTIES AND METHODS FOR UV TREATMENT”, filed by UV Partners, Inc., on Mar. 6, 2020, which is incorporated herein by reference in its entirety. Although the systems and methods set forth in WO 2021/080638A1 are described primarily in the context of controlling the distribution of UV light, they can be used in connection with the present invention to control the distribution of visible light and/or UVC light from the backlight screen diffuser. Some options for allowing light to uniformly escape the diffuser 704 into the adjacent LCD may include the application of a surface treatment to the major surface of the diffuser facing the LCD (i.e., the surface through which the transmission of light is desired). For example, the front major surface of the diffuser may be frosted or receive an alternative surface treatment that facilitates the passage of light through the surface in a substantially uniform manner. Alternative surface treatments may include a blocking pattern that is provided over the front face of the diffuser to control light distribution. In some embodiments, the blocking pattern includes a pattern of reflective regions (e.g., reflective dots) that reflect light back into the diffuser. The blocking pattern may be progressively less dense at increasing distances from the parabolic reflector 706. Generally, the properties of the diffuser material can be selectively controlled by loading the diffuser 704 with additives, applying different blocking patterns to the surface through which light is to be transmitted, varying the material type, thickness, shape, layering, surface texture, or by the application of transmissive films, or any combination thereof as set forth herein or in WO 2021/080638A1. Further, if desired, the major surface of the diffuser opposite the LCD (i.e., the surface through which light transmission is not desired) may be covered by a reflector or a reflective coating that reduces or eliminates the escape of light through the back surface.

Both frontlighting and backlighting applications can be implemented using:
A single parabolic reflector with a light source on one end of the reflector.
A single parabolic reflector with a light source on both ends of the reflector.
Multiple parabolic reflectors that each include a light source to illuminate a portion of the LCD screen.
Channel lights that have a special piece at the “end” of the channel, which reflects any remaining energy towards the target screen using a more traditional reflector.

Although various LCD illumination systems have been described in connection with the distribution of visible light, a single light engine configured to provide frontlighing/backlighting can be configured to also disinfect the surface when using transmissive materials. For example, the channel lighting system may include separate visible and UVC light sources that produce visible and UVC light that is distributed over the surface. In one application, a light source capable of producing visible and UVC light may be positioned at the focal point of the parabolic reflector. In an alternative application, separate visible light and UVC light sources may be positioned as close as possible to focal point at one end of the parabolic reflector. In yet another alternative application, a visible light source may be located at or near the focal point at one end of a parabolic reflector and a UVC light source may be located at or near the focal point at the opposite end of the parabolic reflector. In applications of this type, a reflector may be situated as closely as possible about each light source to reflect back and light from the opposite light source. Those reflectors may be configured to reflect light toward the surface to be illuminated or in essentially any desired direction. In other applications, a first channel lighting system producing visible light may be disposed along one edge of the surface to be illuminated and a second channel lighting system producing UVC light may be located along a second edge of the surface. For example, separate visible and UVC channel lighting systems may be positioned along opposite or adjacent edges of the surface to be illuminated.

Although various low profile light delivery systems have been described in connection with a parabolic shaped anisotropic reflector, it should be understood that parabolic shaped anisotropic reflectors can include parabolic-like shaped reflectors that approximate a true parabolic shaped anisotropic reflector and provide similar or suitable results. For example, certain portions of spherical (circular) and elliptical shaped reflector curvatures are somewhat similar to parabolic shaped reflectors and have somewhat similar properties to that of a parabolic reflector and therefore can be substituted for a parabolic shaped reflectors in various embodiments. This is because the curvature (or portion thereof) of a spherical or elliptical reflector matches fairly closely with the curvature of a parabolic reflector. In some embodiments it may be preferred to utilize an elliptical, spherical (circular), or other shaped anisotropic reflector instead of a parabolic anisotropic reflector.

Directional terms, such as “vertical,” “horizontal,” “top,” “bottom,” “upper,” “lower,” “inner,” “inwardly,” “outer” and “outwardly,” are used to assist in describing the invention based on the orientation of the embodiments shown in the illustrations. The use of directional terms should not be interpreted to limit the invention to any specific orientation(s).

The above description is that of current embodiments of the invention. Various alterations and changes can be made without departing from the spirit and broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims. Any reference to claim elements in the singular, for example, using the articles “a,” “an,” “the” or “said,” is not to be construed as limiting the element to the singular.

1. A system and method for disinfecting a display surface:
a low profile delivery pattern limits human exposure to a portion of a fingertip including:
a parabolic reflector with;
at least one UV LED directed at an angle longitudinal to the reflector.

2. The system and method of claim 1 wherein the system provides variable energy based on desired dosage and target LED life.

3. The system and method of claim 1 wherein the system is integrated with network tracking for life and touch systems.

4. The system and method of claim 1 wherein the system identifies cleaning cycles on a surface utilizing sensors using a secure network, collecting sensor data with monitor when enabled; and touch sensing, contact, capacitive, PIR, Motion, resistive.

5. The system and method of claim 1 wherein the system enables health ranking over multiple workflow uses.

6. A system for UV delivery to a target surface of a touch display, the system comprising:
an anisotropic reflective UV channel configured to mount along an edge a display;
one or more UV LEDs configured to project UV light toward the anisotropic reflective UV channel at a predetermined angle to the channel;
a variable UV LED driver for driving power to the one or more UV LEDs;
one more sensors for sensing;
a controller configured to control the UV LED driver to provide generally uniform UV intensity at the target surface.

7. The system of claim 6 wherein the uniform UV intensity is provided between 2 uW/cm² and 10 uW/cm².

8. The system of claim 6 integrated with a kiosk computer, kiosk touch interface, and kiosk power system.

9. The system of claim 6 including a communications interface for communicating with a cloud database configured to store UV delivery system information.

10. The system of claim 6 wherein the controller is configured to respond to sensor output from the sensors to provide for one or more of proximity based shutoff, movement shutoff, and maintenance shutoff.

11. The system of claim 6 including two or more UV LEDs configured to project UV light toward the anisotropic reflective UV channel at a predetermined angle to the channel, and wherein the controller is configured to control activation and power level to the UV variable LED driver to position UV energy on the target surface of the display within a quadrant of the target surface.

12. The system of claim 6 including two or more UV LEDs configured to project UV light toward the anisotropic reflective UV channel at a predetermined angle to the channel, and wherein the controller is configured to control activation and power level to the UV variable LED driver to position UV energy on a portion of the target surface of the display.

13. The system of claim 6 wherein the anisotropic reflective UV channel is parabolic.

14. The system of claim 6 wherein the anisotropic reflective UV channel is U-shaped including two side walls connected by a bottom wall, wherein the bottom wall is diffuse reflector and the two side walls are specular reflectors.

15. The system of claim 14 wherein the bottom wall is a 94% reflective diffuse reflector and the two side walls are each 90% reflective specular reflectors.

16. The system of claim 6 including a UV transmissive window and protective louvers.

17. The system of claim 6 wherein the anisotropic reflective UV channel includes diffuse channels perpendicular to the longitudinal axis of the reflector.

18. The system of claim 6 wherein the anisotropic reflective UV channel includes selectively diffuse portions and selectively specular portions configured to provide homogenous UV intensity over the target surface.

19. The system of claim 18 wherein the anisotropic reflective UV channel is configured according to a ratio between diffuse and specular portions.

20. The system of claim 19 wherein the area and ratio between diffuse and specular portions provides a longitudinal pattern of the anisotropic reflective UV channel.

21. The system of claim 19 wherein the area and ratio between diffuse and specular portions provides a radial pattern of the anisotropic reflective UV channel.

22. The system of claim 6 wherein the anisotropic reflective UV channel is segmented.

23. The system of claim 22 wherein a selected segmented ratio between diffuse, specular, and orientation shape contribute to provide geometry and optical properties of the segmented anisotropic reflective UV channel.

24-67. (canceled)
